(19) United States
(12) Patent Application Publication
(10) Pub. No.: US 2025/0041228 A1
(43) Pub. Date: Feb. 6, 2025

(54) ORAL FORMULATION OF A PYRIDINONE DERIVATE AND USE THEREOF IN PROPHYLAXIS AND/OR TREATMENT OF INTESTINAL FIBROSIS

(71) Applicant: Dr. Falk Pharma GmbH, Freiburg (DE)

(72) Inventors: Rudolf Wilhelm, Bischweier (DE); Bernhard Tewes, Vörstetten (DE); Roland Greinwald, Kenzingen (DE)

(21) Appl. No.: 18/704,228

(22) PCT Filed: Oct. 24, 2022
(86) PCT No.: PCT/EP2022/079666

§ 371 Date: Apr. 24, 2024

(30) Foreign Application Priority Data
Oct. 25, 2021 (WO) PCT/EP2021/079584

(51) Int. Cl. A61K 9/20; A61K 9/28; A61K 9/50; A61K 31/4439

(52) U.S. Cl. CPC A61K 9/2081; A61K 9/2866; A61K 9/501; A61K 9/5015; A61K 9/5026; A61K 9/5042; A61K 9/5078; A61K 31/4439

(57) ABSTRACT

The present invention relates to an oral formulation adapted for selective delivery of a drug to the small intestine of a mammal comprising or consisting of: a plurality of particles in form of coated granules, coated pellets, coated beads, coated minicapsules, or coated minitablets, each particle containing (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof as the drug, a core material, and an enteric coating polymer, wherein each particle is coated with at least one coating layer containing or consisting of the enteric coating polymer; a preparation method of said oral formulation; and a medical use of said oral formulation in prophylaxis or treatment of intestinal fibrosis, in particular, fibrostenotic Crohn's disease.

The present invention relates to an oral formulation adapted for selective delivery of a drug to the small intestine of a mammal comprising or consisting of: a plurality of particles in form of coated granules, coated pellets, coated beads, coated minicapsules, or coated minitablets, each particle containing (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof as the drug, a core material, and an enteric coating polymer, wherein each particle is coated with at least one coating layer containing or consisting of the enteric coating polymer; a preparation method of said oral formulation; and a medical use of said oral formulation in prophylaxis or treatment of intestinal fibrosis, in particular, fibrostenotic Crohn's disease.

### BACKGROUND OF THE INVENTION

Intestinal fibrosis is a common and potentially serious complication of inflammatory bowel disease (IBD) that results from the reaction of intestinal tissue to the damage inflicted by chronic inflammation. The traditional view that fibrosis is inevitable or irreversible in patients with IBD is progressively changing in light of improved understanding of the cellular and molecular mechanisms that underlie the pathogenesis of fibrosis in general, and, in particular, intestinal fibrosis.

Intestinal fibrosis is a common but debilitating complication of Crohn's disease for which currently there is no medical treatment. Crohn's disease (CD) is a form of inflammatory bowel disease, which results in areas of chronic inflammation at any point in the gastrointestinal tract. In particular Crohn's disease mainly affects the small and large intestine.

In a number of cases, significant fibrosis is already present at the time of disease diagnosis, and 40-70% of Crohn's disease patients require surgery in the 10 years following diagnosis, with the development of fibrotic structures being the leading indication for surgery in small bowel disease. Fibrosis occurs due to excessive deposition of extracellular matrix (ECM), particularly fibrous collagen, and dysregulated turnover of ECM.

It is known that Transgluaminase 2 (TG2) is activated in the inflamed intestine of IBD patients, as significantly high levels of TG2 autoantibody have been detected in the serum of Crohn's disease. (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I) is an effective inhibitor against transglutaminase 2.

However, it is still a problem to design an appropriate pharmaceutical formulation for the oral route of gastrointestinal drug delivery, in particular, selective local drug release into the small intestine to treat intestinal fibrosis, in particular, fibrostenotic Crohn's disease.

It is the objective of the present invention to provide an oral formulation comprising (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof as a drug adapted for selective delivery of said drug to the small intestine of a mammal for the prophylaxis or treatment of intestinal fibrosis, in particular, fibrostenotic Crohn's disease.

The objective of the present invention is solved by the teaching of the independent claims. Further advantageous features, aspects and details of the invention are evident from the dependent claims, the description, the FIGURES, and the examples of the present application.

### BRIEF DESCRIPTION OF THE INVENTION

The objective of the present invention is solved by an oral formulation adapted for selective delivery of a drug to the small intestine of a mammal comprising or consisting of:
a plurality of particles in form of coated granules, coated pellets, coated beads, coated minicapsules, or coated minitablets, each particle containing (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I):

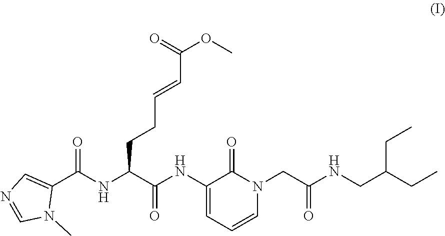 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof as the drug, a core material, and an enteric coating polymer, wherein each particle is coated with at least one coating layer containing or consisting of the enteric coating polymer.

Herein the compound of the formula (I) is also referred to as Compound 1 or Comp. 1.

Preferably, in the oral formulation of the present invention the enteric coating polymer has an average molecular mass>100,000 g/mol and is selected from methacrylic acid—methacrylic acid ester copolymers, acrylic acid-methacrylic acid ester copolymers, methacrylic acid-acrylic acid ester copolymers, acrylic acid-acrylic acid ester copolymers, acrylic acid ester-methacrylic acid ester-methacrylic acid copolymers, wherein the ester is a methyl ester, ethyl ester, propyl ester, iso-propyl ester, or butyl ester and preferably a methyl or ethyl ester, and most preferably a methyl ester.

More preferably the enteric coating polymer is selected from methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and a mixture of two or more of said enteric coating polymers.

Still more preferably the enteric coating polymer is selected from the group comprising or consisting of: hypromellose acetate succinate (HPMCAS), hypromellose phthalate, methacrylic acid-methyl methacrylate copolymers such as Eudragit® L100, Eudragit® L12.5, Eudragit® L12.5P, Eudragit® L30D-55, Eudragit® L100-55, Eudragit® S100, Eudragit® S12.5, Eudragit® S12.5P, Eudragit® RS100, Eudragit® RL100, Eudragit® RL12.5, Eudragit® RS12.5, Eudragit® FS30D, methacrylic acid-ethyl acrylate copolymers, methyl acrylate-methyl methacrylate-methacrylic acid copolymers, polyvinyl acetate phthalate, cellulose acetate phthalate (CAP), shellac and a mixture of two or more of said enteric coating polymers.

More preferably, the hypromellose acetate succinate is selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; the methacrylic acid-methyl methacrylate copolymer is selected from the group consisting of: Eudragit® L100, Eudragit® S100, and a mixture thereof.

The core material is preferably in form of a core pellet and is preferably selected from the group comprising or consisting: tartaric acid, lactose, sugar, maize starch, starch hydrolysates, silica, or microcrystalline cellulose, and preferably, microcrystalline cellulose.

In addition, the present invention refers to the oral formulation as mentioned above, wherein each particle is coated in addition to the enteric coating comprising or consisting of the enteric coating polymner with a further coating layer comprising or consisting of a sustained-release polymer. This sustained-release layer contains or consists of the sustained-release polymer which is preferably selected from hypromellose (HPMC), ethylcellulose, ammonio methacrylate co-polymers such as Eudragit® RL100 and Eudragit® RS100, Eudragit® RL12.5, Eudragit® RS12.5, and a mixture thereof.

In case the sustained-release polymer is ethylcellulose, optionally polyethylene glycol (PEG) is further comprised as plasticizer, preferably Macrogol® 6000.

It is found that the oral formulation of the present invention starts to release (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof in the jejunum and completing the release in the ileum.

Furthermore, the oral formulation of the present invention is useful in the prophylaxis and/or treatment of intestinal fibrosis, in particular, fibrostenotic Crohn's disease.

The oral formulation of the present invention is produced by the following method comprising:
Step A) preparing a plurality of particles in form of coated granules, coated pellets, coated beads, coated minicapsules, or coated minitablets, wherein each particle contains
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I)
   or an enantiomer, a solvate, a hydrate or a pharmaceutically acceptable salt thereof; and
   a core material;
Step B) coating each particle with at least one coating solution comprising an enteric coating polymer.

Optionally, the inventive method further comprises:
Step C) coating each particle with a coating solution comprising sustained-release polymer.

### DESCRIPTION OF THE INVENTION

The present invention relates to an oral formulation adapted for selective delivery of a drug to the small intestine of a mammal comprising or consisting of:
a plurality of particles in form of coated granules, coated pellets, coated beads, coated minicapsules, or coated minitablets, each particle containing (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I):

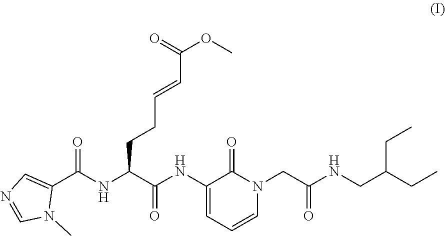 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof, a core material, and an enteric coating polymer, wherein each particle is coated with at least one coating layer containing or consisting of the enteric coating polymer.

As used herein, the term “oral formulation” refers to a formulation being a medication which is administered through the mouth.

As used herein, “selective delivery of a drug to the small intestine” means that the formulation is gastro-resistant and can resist the fluid in the stomach and release the active ingredient in the intestine.

As used herein, “a mammal” refers to an animal, or a human, preferably a patient suffering from intestinal fibrosis, in particular fibrostenotic Crohn's disease.

As used herein, the term “pharmaceutically acceptable salts” refers to salts of certain ingredient(s) which possess the same activity as the unmodified compound(s) and which are neither biologically nor otherwise undesirable.

A pharmaceutically acceptable salt can be formed with, for example, organic or inorganic acids. Suitable acids include acetic acid, acetylsalicylic acid, organic di-carboxylic acid such as oxalic acid, malonic acid, succinic acid, glutaric acid, tartaric acid, fumaric acid, maleic acid, malic acid, adipic acid, or glutamic acid, and organic tri-carboxylic acid such as citric acid, or sodium hydrogen citrate alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

As used herein, the term “solvates” refers to those forms of a compound in particular the (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate which form a complex through coordination with solvent molecules.

As used herein, the term “hydrates” refers to those forms of a compound in particular the (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate which form a complex through coordination with water molecules.

As used herein, the term “effective amount” or “therapeutically effective amount” of an active agent or a pharmaceutically active agent or a drug or an active pharmaceutical ingredient, which are synonymous herein, refers to an amount of the active agent or pharmaceutically active agent or drug or active pharmaceutical ingredient, sufficient enough to have a positive effect. Accordingly, these amounts are sufficient to treat the intended diseases, but still sufficiently low to avoid serious side effects. A therapeutically effective amount of the pharmaceutically active agent will cause a substantial relief of symptoms when applied repeatedly over time. Effective amounts of the pharmaceutically active agent will vary with the particular condition or conditions being treated, the severity of the condition, the duration of the treatment, the specific components of the composition being used, and like factors.

As used herein, the term “active agent”, “pharmaceutically active agent”, “drug” or “active pharmaceutical ingredient”, which are synonymously used herein, refers to a compound exhibiting a therapeutic effect upon a mammal in particular a human.

As used herein, the “enteric coating polymer” refers to a polymer applied to oral medication that prevents its dissolution or disintegration in the gastric environment, and helps by either protecting drugs from the acidity of the stomach, the stomach from the detrimental effects of the drug, and to release the drug after the stomach usually in the intestine, preferably in the small intestine. Granules, pellets, beads, minicapsules and minitablets are preferred enteric-polymer-coated dosage forms in the present invention.

Preferably, the oral formulation of the present invention comprises the enteric coating polymer in a range of 1 wt % to 40 wt %, preferably 1 wt % to 35 wt %, more preferably 1 wt % to 30 wt %, most preferably 1 wt % to 26 wt %.

The highly acidic gastric environment (pH 1.5-2 in the fasted state) rises rapidly to pH 6 in the duodenum and increases along the small intestine to pH 7.4 at the terminal ileum.

Hence, the oral formulation should be retained in the acidic gastric environment and can be dissolved in the small intestine.

Thus, the oral formulation of the present invention comprises the enteric coating polymer and the enteric coating polymer is dissolved at a pH-value in a range of 5.0 to 7.5, preferably 5.0 to 7.0, more preferably 5.5 to 7.0, most preferably 5.5 to 6.8.

Preferably, the eneric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

Thus, in some embodiments, the invention is directed to the oral formulation adapted for selective delivery of a drug to the small intestine of a mammal comprising or consisting of:
a plurality of particles in form of coated granules, coated pellets, coated beads, coated minicapsules, or coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, a core material, an enteric coating polymer, and
      wherein each particle is coated with at least one coating layer containing or consisting of the enteric coating polymer; and
      the eneric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

Hypromellose is also known as Hydroxypropylmethylcellulose (HPMC), and thus hypromellose acetate succinate (hydroxypropyl methylcellulose acetate succinate, or HPMCAS) is acetic acid/succinic acid ester of hydroxypropyl methylcellulose.

Hypromellose phthalate (hydroxypropyl methylcellulose phthalate, or HPMCP) is a phthalic acid ester of hydroxypropyl methylcellulose.

Hypromellose (HPMC), hypromellose acetate succinate (HPMCAS), and hypromellose phthalate (HPMCP) have the following chemical structure respectively:

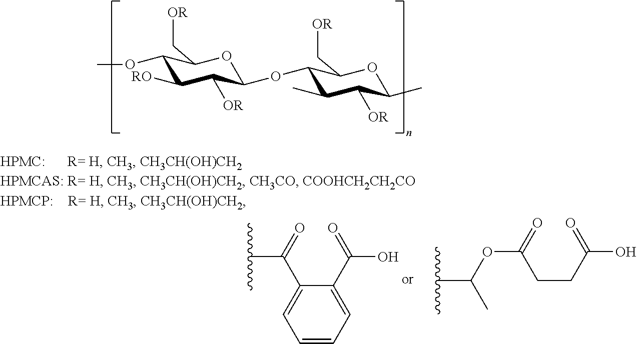

HPMCAS is a cellulosic polymer with four types of substituents semirandomly substituted on the hydroxyls: methoxy, with a mass content of 12-28 wt %; hydroxypropyl, with a mass content of 4-23 wt %; acetate, with a mass content of 2-16 wt %; and succinate, with a mass content of 4-28 wt %. The succinate groups of HPMCAS have a pKa of about 5, and therefore, the polymer is less than 10% ionized at pH values below about 4 and is at least 50% ionized at pH values of about 5 or higher. Due to the presence of relatively hydrophobic methoxy and acetate substituents, HPMCAS is water-insoluble when un-ionized (about pH<5) and remains predominantly colloidal at intestinal pH (that is, pH 6.0-7.5).

There are nine grades with different particle sizes (fine, medium, granular) of HPMCAS and chemical substitution levels of acetyl and succinoyl groups to obtain an opening pH range from 5.5 to 6.5 (Shin-Etsu AQOAT® Enteric Coating Agent, or Ashland AquaSolve™).

| | | Hydroxy- | | | | |
|---|---|---|---|---|---|---|
| | Methoxy | propoxy | Acetyl | Succinyl | | pH |
| Grade | (%) | (%) | (%) | (%) | Particle | Solubility |
| LF | 20-24 | 5-9 | 5-9 | 14-18 | Fine^{a)} | ≥5.5 |
| LMP | 20-24 | 5-9 | 5-9 | 14-18 | Medium^{b)} | ≥5.5 |
| LG | 20-24 | 5-9 | 5-9 | 14-18 | Granular | ≥5.5 |
| MF | 21-25 | 5-9 | 7-11 | 10-14 | Fine^{a)} | ≥6.0 |
| MMP | 21-25 | 5-9 | 7-11 | 10-14 | Medium^{b)} | ≥6.0 |
| MG | 21-25 | 5-9 | 7-11 | 10-14 | Granular | ≥6.0 |
| HF | 22-26 | 6-10 | 10-14 | 4-8 | Fine^{a)} | ≥6.5 |
| HMP | 22-26 | 6-10 | 10-14 | 4-8 | Medium^{b)} | ≥6.5 |
| HG | 22-26 | 6-10 | 10-14 | 4-8 | Granular | ≥6.5 |

^{a)}D₅₀; NMT 10 μm, D₉₀: NMT20 μm.;
^{b)}D₅₀: 70-300 μm

In some preferred embodiments, the oral formulations as described herein, the enteric coating polymer is the hypromellose acetate succinate (HPMCAS) and said HPMCAS is selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF.

Thus, the invention refers to the oral formulation adapted for selective delivery of a drug to the small intestine of a mammal comprising or consisting of:
a plurality of particles in form of coated granules, coated pellets, coated beads, coated minicapsules, or coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, a core material, an enteric coating polymer, and
wherein each particle is coated with at least one coating layer containing or consisting of the enteric coating polymer; and
   the enteric coating polymer is the hypromellose acetate succinate (HPMCAS) and said HPMCAS is selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF.

Cellulose acetate phthalate (CAP), also known as cellacefate (INN), is a cellulose polymer where about half of the hydroxyls are esterified with acetyls, a quarter are esterified with one or two carboxyls of a phthalic acid, and the remainder are unchanged. CAP has the following chemical structure:

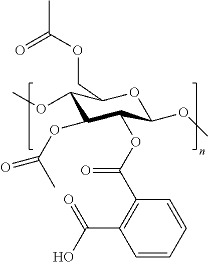

The term “(meth)acrylic acid-(meth)acrylate copolymers” refers to copolymers of acrylic acid or acrylic acid ester and methacrylic acid or methacrylic acid ester. Such copolymers have a molecular mass (abbreviated Mr), formerly also called molecular weight of Mᵣ≥100 000 and have the following general formula:

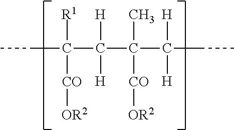

wherein
R¹ is most preferably —H or —CH₃; and
R² and R³ are independently of each other —H or an ester group, preferably a methyl, ethyl, propyl, iso-propyl, or butyl ester.

Preferred are the Eudragit® copolymers such as Eudragit® L100, Eudragit® L12.5, Eudragit® L12.5P, Eudragit® L30D-55, Eudragit® L100-55, Eudragit® S100, Eudragit® S12.5, Eudragit® S12.5P, Eudragit® RS100, Eudragit® RL100, Eudragit® RL12.5, Eudragit® RS12.5, Eudragit® FS30D and a mixture of two or three of these Eudragit® copolymers, more preferred are Eudragit® L100, Eudragit® S100, and a mixture thereof. Eudragit® S100, Eudragit® S12.5, Eudragit® S12.5P (Evonik) have a ratio of methacylic acid:methyl metacrylate=1:2 and a mean relative molecular mass of about 135 000. The ratio of carboxylic groups to ester groups is about 1:2. Between 25% and 35% of the residues R² and R³ are —H in the Eudragit® S copolymers, that means between 25% and 35% of the carbonyloxy moieties are carboxyl groups (—COOH). The content of carboxyl groups in the Eudragit® S copolymers is preferably 30%±1%.

Eudragit® L100, Eudragit® L12.5, Eudragit® L12.5P (Evonik) have a ratio of methacylic acid:methyl metacrylate=1:1 and a mean relative molecular mass of about 250000. The ratio of carboxylic groups to ester groups is about 1:1.

Eudragit® L100-55 and Eudragit® L30D-55 (Evonik) have a ratio of methacrylic acid ethylacrylate=1:1 and a mean relative molecular mass of about 250000. The ratio of carboxylic groups to ester groups is about 1:1.

Between 45% and 55% of the residues R² and R³ are —H in the Eudragit® L copolymers, that means between 45% and 55% of the carbonyloxy moieties are carboxyl groups (—COOH). The content of carboxyl groups in the Eudragit® L copolymers is preferably 50%±1%.

Eudragit® FS30D (Evonik) have a ratio of methyl acrylate, methyl methacrylate, and methacrylic acid of 7:3:1 and a mean average molar mass of 280 000 g/mol. The ratio of the free carboxyl groups to the ester groups is about 1:10.

These polymers are gastroresistant and enterosoluble polymers. Their polymer films are insoluble in pure water and diluted acids. They dissolve at higher pHs, depending on their content of carboxylic acid. Eudragit® S copolymers and Eudragit® L copolymers can be used as single components in the polymer coating or in combination in any ratio. By using a combination of the polymers, the polymeric material can exhibit solubility at a pH between the pHs at which Eudragit® L copolymers and Eudragit® S copolymers are separately soluble.

In the embodiments of the oral formulations as described herein, the enteric coating polymer is a methacrylic acid-methacrylic acid ester copolymer, a acrylic acid-methacrylic acid ester copolymer, a methacrylic acid-acrylic acid ester copolymer, a acrylic acid-acrylic acid ester copolymer, and more preferably a methacrylic acid-methyl methacrylate copolymer, a acrylic acid-methyl methacrylate copolymer, a methacrylic acid-methyl acrylate copolymer, a acrylic acid-methyl acrylate copolymer, still more preferably Eudragit® L100, Eudragit® L12.5, Eudragit® L12.5P, Eudragit® L30D-55, Eudragit® L100-55, Eudragit® S100, Eudragit® S12.5, Eudragit® S12.5P, Eudragit® FS30D and a mixture of two or three of these Eudragit® copolymers, and most preferably Eudragit® L100, Eudragit® S100, or a mixture thereof.

Eudragit® L100-x:y=1:1 (ratio of metacrylic acid:methyl metacrylate units);

Eudragit® S100-x:y=1:2 (ratio of metacrylic acid:methyl metacrylate units);

n=degree of polymerization.

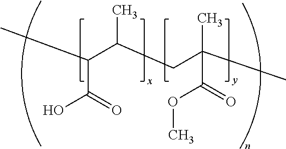

Preferably, the invention refers to an oral formulation adapted for selective delivery of the drug (i.e. Comp. 1) to the small intestine of a mammal comprising or consisting of:
a plurality of particles in form of coated granules, coated pellets, coated beads, coated minicapsules, or coated minitablets, each particle containing:
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate as the drug, or an enantiomer, a solvate, a hydrate or a pharmaceutically acceptable salt thereof, and
   a core material, and
   an enteric coating polymer,
      wherein each particle is coated with at least one coating layer containing or consisting of the enteric coating polymer; and
      the enteric coating polymer is selected from hypromellose acetate succinate (HPMCAS), hypromellose phthalate, polyvinyl acetate phthalate, cellulose acetate phthalate (CAP), shellac; or
      has an average molecular mass>100,000 g/mol, preferably an average molecular mass between 120,000 g/mol and 270,000 g/mol and is represented by the following general formula:

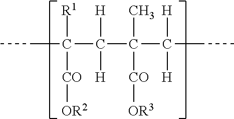

wherein
R¹ is —H or —CH₃; and
R² is —H and R³ is selected from methyl, ethyl, propyl, iso-propyl, butyl or dimethylaminoethyl or
R³ is —H and R² is selected from methyl, ethyl, propyl, iso-propyl, or butyl or a mixture of one or more of these enteric coating polymers;
more preferably the enteric coating polymer is a methacrylic acid-methacrylic acid ester copolymer, a acrylic acid-methacrylic acid ester copolymer, a methacrylic acid-acrylic acid ester copolymer, a acrylic acid-acrylic acid ester copolymer, a acrylic acid ester-methacrylic acid ester-methacrylic acid copolymer, wherein the ester is a methyl ester, ethyl ester, propyl ester, iso-propyl ester, or butyl ester, more preferably a methyl ester or an ethyl ester or a mixture thereof, and most preferably a methyl ester,
thus, still more preferably the enteric coating polymer is selected from hypromellose acetate succinate (HPMCAS), hypromellose phthalate, polyvinyl acetate phthalate, cellulose acetate phthalate (CAP), shellac, methacrylic acid-methyl methacrylate copolymer, acrylic acid-methyl methacrylate copolymer, methacrylic acid-methyl acrylate copolymer, methacrylic acid-ethyl acrylate copolymer, acrylic acid-methyl acrylate copolymer, methyl acrylate-methyl methacrylate-methacrylic acid copolymer or a mixture of one or more of these enteric coating polymers;
still more preferably the enteric coating polymer has an average molecular mass>100,000 g/mol, preferably an average molecular mass between 120,000 g/mol and 270,000 g/mol and is represented by one of the following two general formulae:

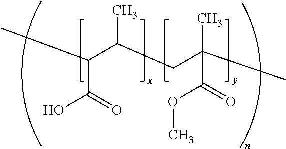

wherein
n=degree of polymerization
and the ratio of x:y is in the range of 1:0.5 to 1:2.5, preferably 1:0.8 to 1:2.2;
or

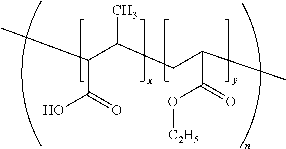

wherein
n=degree of polymerization
and the ratio of x:y is in the range of 1:0.5 to 1:2.5, preferably 1:0.8 to 1:2.2;
still more preferably the enteric coating polymer is selected from the group comprising or consisting of:
Eudragit® L100, Eudragit® L12.5, Eudragit® L12.5P, Eudragit® L30D-55, Eudragit® L100-55, Eudragit® S100, Eudragit® S12.5, Eudragit® S12.5P, Eudragit® FS30D, hypromellose acetate succinate (HPMCAS), hypromellose phthalate, polyvinyl acetate phthalate, cellulose acetate phthalate (CAP), shellac and a mixture of two or three of these Eudragit® copolymers.

The methacrylate co-polymers can be combined in any desired ratio, and the ratio can be modified to modify the rate of drug release. A ratio of Eudragit® L100:Eudragit® S100 can be in a range of 100:0 to 50:50, preferably 100:0 to 60:40, 100:0 to 70:30, more preferably 100:0 to 80:20, or any ratio in between, most preferably, a ratio of Eudragit® L100:Eudragit® S100 is 100:0 to 85:15.

In all embodiments disclosed herein, the core material is preferably in form of a core pellet or a granule and is preferably selected from the group comprising or consisting: tartaric acid, lactose, sugar, maize starch, starch hydrolysates, silica, or microcrystalline cellulose, and preferably, microcrystalline cellulose.

The term “core material in form of a core pellet” means that core pellets are prepared which consist only of the core material or a mixture of the cited core materials. These core pellets do not contain the drug (i.e. Comp. 1) and do not contain the enteric coating polymer or a sustained-release polymer. These core pellets are coated with a drug containing layer, an enteric coating layer and optionally a sustained-release layer. Instead of the term “layer”, the term “coating” could alternatively be used. If present the sustained-release layer could be on top of the enteric coating layer or below the enteric coating layer.

The term “core material in form of a granule” means that the core material or a mixture of the cited core materials as main component(s) together with the drug and optionally further components are mixed together to obtain a composition of which granules are formed. These granules contain Comp. 1 most probably in a homogeneously distributed manner and are coated with an enteric coating layer and optionally a sustained-release layer. If present the sustained-release layer could be on top of the enteric coating layer or below the enteric coating layer.

Consequently, the invention refers to an oral formulation adapted for selective delivery of the drug (i.e. Comp. 1) to the small intestine of a mammal comprising or consisting of:
a plurality of particles in form of coated granules, coated pellets, coated beads, coated minicapsules, or coated minitablets, each particle containing:
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate as the drug, or an enantiomer, a solvate, a hydrate or a pharmaceutically acceptable salt thereof, and
   a core material, and
   an enteric coating polymer,
      wherein the core material is selected from: tartaric acid, lactose, sugar, maize starch, starch hydrolysates, silica, or microcrystalline cellulose, and preferably, microcrystalline cellulose; and
      wherein each particle is coated with at least one coating layer containing or consisting of the enteric coating polymer; and
      the enteric coating polymer is selected from hypromellose acetate succinate (HPMCAS), hypromellose phthalate, polyvinyl acetate phthalate, cellulose acetate phthalate (CAP), shellac or
      has an average molecular mass>100,000 g/mol, preferably an average molecular mass between 120,000 g/mol and 270,000 g/mol and is represented by the following general formula:

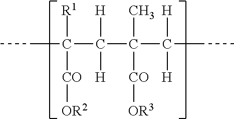

wherein
R¹ is —H or —CH₃; and
R² is —H and R³ is selected from methyl, ethyl, propyl, iso-propyl, butyl or dimethylaminoethyl or
R³ is —H and R² is selected from methyl, ethyl, propyl, iso-propyl, or butyl or a mixture of one or more of these enteric coating polymers;
more preferably the enteric coating polymer is a methacrylic acid-methacrylic acid ester copolymer, a acrylic acid-methacrylic acid ester copolymer, a methacrylic acid-acrylic acid ester copolymer, a acrylic acid-acrylic acid ester copolymer, a acrylic acid ester-methacrylic acid ester-methacrylic acid copolymer, wherein the ester is a methyl ester, ethyl ester, propyl ester, iso-propyl ester, or butyl ester, more preferably a methyl ester or an ethyl ester or a mixture thereof, and most preferably a methyl ester, thus, still more preferably the enteric coating polymer is selected from hypromellose acetate succinate (HPMCAS), hypromellose phthalate, polyvinyl acetate phthalate, cellulose acetate phthalate (CAP), shellac, methacrylic acid-methyl methacrylate copolymer, acrylic acid-methyl methacrylate copolymer, methacrylic acid-methyl acrylate copolymer, methacrylic acid-ethyl acrylate copolymer, acrylic acid-methyl acrylate copolymer, methyl acrylate-methyl methacrylate-methacrylic acid copolymer or a mixture of one or more of these enteric coating polymers;
still more preferably the enteric coating polymer has an average molecular mass>100,000 g/mol, preferably an average molecular mass between 120,000 g/mol and 270,000 g/mol and is represented by one of the following two general formulae:

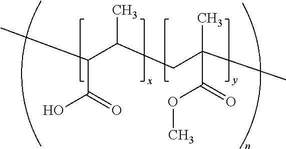

wherein
n=degree of polymerization
and the ratio of x:y is in the range of 1:0.5 to 1:2.5, preferably 1:0.8 to 1:2.2; or

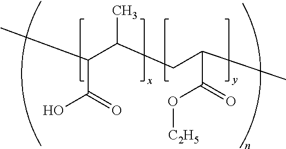

wherein
n=degree of polymerization
and the ratio of x:y is in the range of 1:0.5 to 1:2.5, preferably 1:0.8 to 1:2.2; still more preferably the enteric coating polymer is selected from the group comprising or consisting of:
Eudragit® L100, Eudragit® L12.5, Eudragit® L12.5P, Eudragit® L30D-55, Eudragit® L100-55, Eudragit® S100, Eudragit® S12.5, Eudragit® S12.5P, Eudragit® FS30D, hypromellose acetate succinate (HPMCAS), hypromellose phthalate, polyvinyl acetate phthalate, cellulose acetate phthalate (CAP), shellac and a mixture of two or three of these Eudragit® copolymers.

Thus, more preferably, the invention refers to the oral formulation adapted for selective delivery of a drug to the small intestine of a mammal comprises or consists of:
a plurality of particles in form of coated granules, coated pellets, coated beads, coated minicapsules, or coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically
   acceptable salt thereof,
   a core material, and
   an enteric coating polymer,
wherein each particle is coated with at least one coating layer containing or consisting of the enteric coating polymer; and
the enteric coating polymer is the methacrylic acid-methyl methacrylate copolymer and the methacrylic acid-methyl methacrylate copolymer is Eudragit® L100, Eudragit® S100, or a mixture thereof.

The mixture of Eudragit® L100, Eudragit® S100 may have a ratio in a range of 100:0 to 50:50, preferably 95:5 to 60:40, 90:10 to 70:30, more preferably 90:10 to 80:20, or any ratio in between, most preferably, a ratio of Eudragit® L100:Eudragit® S100 is 85:15.

After the administration of the oral formulation of the present invention, the administered drug dose has to dissolve quickly and completely. The pH variations in the stomach after the oral administration have to be regulated and it should be ensured that the administered drug dose is dissolved. The bigger the systemic availability (AUC) in conjunction with the mucosal release in the small intestine, the higher pharmacological effect to be expected.

In some embodiments, in the oral formulation as described above, each particle further comprises one or more binder(s), buffering agent(s), colorant(s), glidant(s), plasticiser(s), disintegrants(s), pH-modifier(s), surfactant(s) and/or filler(s).

In all embodiments disclosed herein the enteric coating layer contains or consists of the enteric coating polymer.

Moreover, it is preferred that the enteric coating polymer is contained in or forms the outer layer of the oral formulations disclosed herein.

Furthermore, it is preferred that compound 1 is either contained in the core material without an additional drug (i.e. Comp. 1) layer or not in the core material and only in a drug layer covering the core material.

The enteric coating layer covers the core material or the drug layer. However, a further sustained-release layer might be present for the fine-tuning of the release of compound 1 as outermost layer on the enteric coating layer or below the enteric coating layer but above the drug layer or the drug containing core.

In some embodiments, in the oral formulation as described above, the enteric coating polymer is contained in or forms the outer layer of the coating enclosing the core material.

Thus, the invention refers to the oral formulation adapted for selective delivery of a drug to the small intestine of a mammal comprising or consisting of:
a plurality of particles in form of coated granules, coated pellets, coated beads, coated minicapsules, or coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, a core material, an enteric coating polymer, and the core material comprises one or more binder(s), buffering agent(s), colorant(s), plasticiser(s), glidant(s), disintegrants(s), pH-modifier(s), surfactant(s) and/or filler(s),
   wherein each particle is coated with at least one coating layer containing or consisting of the enteric coating polymer.

Preferably, the oral formulation adapted for selective delivery of a drug to the small intestine of a mammal comprises or consists of:
a plurality of particles in form of coated granules, coated pellets, coated beads, coated minicapsules, or coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, a core material, an enteric coating polymer, and the core material comprises one or more binder(s), buffering agent(s), colorant(s), plasticiser(s), glidant(s), disintegrants(s), pH-modifier(s), surfactant(s) and/or filler(s),
   wherein each particle is coated with at least one coating layer containing or consisting of the enteric coating polymer and the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers

More preferably, said oral formulation adapted for selective delivery of a drug to the small intestine of a mammal comprises or consists of:
a plurality of particles in form of coated granules, coated pellets, coated beads, coated minicapsules, or coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   a core material, and the core material comprises one or more binder(s), buffering agent(s), colorant(s), plasticiser(s), glidant(s), disintegrants(s), pH-modifier(s), surfactant(s) and/or filler(s),
   an enteric coating polymer,
      wherein each particle is coated with at least one coating layer containing or consisting of the enteric coating polymer, and of the enteric coating polymer is hypromellose acetate succinate selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100 and a mixture thereof.

In some embodiments, the invention is directed to the oral formulation, wherein the enteric coating polymer is contained in or forms the outer layer of the coating enclosing the core material.

Thus, said oral formulation adapted for selective delivery of a drug to the small intestine of a mammal comprises or consists of:
a plurality of particles in form of coated granules, coated pellets, coated beads, coated minicapsules, or coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, a core material, and an enteric coating polymer,
      wherein each particle is coated with at least one coating layer containing or consisting of the enteric coating polymer and the enteric coating polymer is contained in or forms the outer layer of the coating enclosing the core material.

In addition to granules/pellets/beads multiparticulate formulations also comprise minitablets with typical diameters of one to four millimetres. They are obtained by different manufacturing operations than the spherical particles but they provide the same advantages. They are especially preferred for pediatric applications.

In some embodiments, the invention is directed to the oral formulation, wherein in case the particle is in form of a coated minitablet, the core material comprises one or more binder(s), disintegrants(s), glidant(s), pH-modifier(s), and/or filler(s).

Thus, said oral formulation adapted for selective delivery of a drug to the small intestine of a mammal comprises or consists of:
a plurality of particles in form of coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof;
   a core material, and the core material comprises one or more binder(s), disintegrants(s), glidant(s), pH-modifier(s), and/or filler(s); and
   an enteric coating polymer,
      wherein each particle is coated with at least one coating layer containing or consisting of the enteric coating polymer, preferably, the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers; more preferably, the enteric coating polymer is hypromellose acetate succinate and said hypromellose acetate succinate is selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

Preferably, the binder is selected from the group consisting of: sugar, sucrose, polysaccharides, xanthan gum, guar gum, carrageenan, starches derived from wheat, corn, rice and potatoes, preagglutinated (modified) starch derived from wheat, corn, rice and potatoes, sodium starch glycolate, natural gums, acacia gum, gelatin, tragacanth, derivatives of sea weed, alginic acid, sodium alginate, ammonium calcium alginate, cellulose, cellulose derivatives, hydroxypropyl cellulose, L-hydroxypropyl cellulose, low-substituted hydroxypropyl cellulose, methyl cellulose, sodium carboxymethylcellulose, hydroxypropyl methylcellulose, polyvinylpyrrolidone such as povidone K25, and mixtures thereof.

More preferably, the binder is hydroxypropyl cellulose, L-hydroxypropyl cellulose, low-substituted hydroxypropyl cellulose, or polyvinylpyrrolidone such as povidone K25. Most preferably, the binder is low-substituted hydroxypropyl cellulose or povidone K25.

Thus, said oral formulation adapted for selective delivery of a drug to the small intestine of a mammal comprises or consists of:
a plurality of particles in form of coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, or an enantiomer, a solvate, a hydrate or a pharmaceutically acceptable salt thereof;
   a core material, and the core material comprises one or more binder(s), disintegrants(s), glidant(s), pH-modifier(s), and/or filler(s); and
   the binder is hydroxypropyl cellulose, L-hydroxypropyl cellulose, low-substituted hydroxypropyl cellulose, or polyvinylpyrrolidone such as povidone K25, most preferably the binder is low-substituted hydroxypropyl cellulose or povidone K25.
   and
   an enteric coating polymer,
      wherein each particle is coated with at least one coating layer containing or consisting of the enteric coating polymer.

Hydroxypropyl cellulose is a partially substituted poly(hydroxypropyl) ether of cellulose. It may contain not more than 0.6% of silica or another suitable anticaking agent. Hydroxypropyl cellulose is commercially available in a number of different grades that have various solution viscosities. Molecular weight ranges from 50000-1250000. Hydroxypropylcellulose is partly O-(2-hydroxypropylated) cellulose. It contains 53.4% to 80.5% of hydroxypropoxy groups with reference to the dried substance. The average grade of polymerization ranges from 200 to 300. The molar grade of substitution is around 4.

“Low-substituted hydroxypropyl cellulose” (L-HPC or LHPC) is a low-substituted poly(hydroxypropyl) ether of cellulose. It is commercially available in a number of different grades that have different particle sizes and substitution levels.

Low-substituted hydroxypropyl cellulose contains 5% to 16% hydroxypropoxy groups with reference to the dried substance. The molar grade of substitution is <1. In particular low-substituted hydroxypropyl cellulose is a low-substituted O-(2-hydroxypropylated) cellulose contains hydroxyproxy groups (—OCH₂CHOHCH₃) in a range of 5 wt % to 16 wt″%, preferably 7 wt % to 13 wt % calculated on the dried basis. Low-substituted hydroxypropyl cellulose has a mean particle size in a range of 15 μm to 65 μm, preferably 25 μm to 60 μm, more preferably 25 μm to 60 μm.

“Povidone” is synonymously used for polyvinylpyrrolidone (PVP). Polyvinylpyrrolidone consists of linear polymers of 1-ethenylpyrollidin-2-one. The different types of polyvinylpyrrolidone are characterized by the viscosity of their solutions, expressed by the K value. Polyvinylpyrrolidone is present as a white to yellowish white powder or flake and is readily soluble in water. The K value is a common classification in the plastics industry and is directly related to the average molar mass of the polymer. This makes it possible to deduce indirectly from the K value the degree of polymerization and thus the chain length. Povidone K25, povidone K30 or povidone K90 are commercially available. Preferably, povidone K25 is used as a binder. The approximate average molecular weight of povidone K25 is 30,000 g/mol (Da) between 28,000 g/mol (Da) to 34,000 g/mol (Da).

Preferably, the pH-modifier is selected from the group consisting of: ascorbic acid, oxalic acid, malonic acid, succinic acid, glutaric acid, tartaric acid, fumaric acid, maleic acid, malic acid, adipic acid, glutamic acid, citric acid, and sodium hydrogen citrate, more preferably adipic acid.

A preferred embodiment of the invention is therefore directed to the oral formulation, wherein in case the particle is in form of a coated minitablet, the core material comprises one or more binder(s), disintegrants(s), glidant(s), pH-modifier(s), and/or filler(s), wherein
the binder is low-substituted hydroxypropylcellulose or hydroxypropylcellulose;
the disintegrant is croscarmellose sodium;
the glidant is talc;
the pH-modifier is adipic acid; and/or
the filler is mannitol.

Thus, said oral formulation adapted for selective delivery of a drug to the small intestine of a mammal comprises or consists of:
a plurality of particles in form of coated minitablets, each particle containing:
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof;
   a core material, and the core material comprises one or more binder(s), disintegrants(s), glidant(s), pH-modifier(s), surfactant(s) and/or filler(s),
      wherein the binder is low-substituted hydroxypropylcellulose or hydroxypropylcellulose;
      the disintegrant is croscarmellose sodium;
      the glidant is talc; the pH-modifier is adipic acid; and/or
      the filler is mannitol; and
   an enteric coating polymer,
      wherein each particle is coated with at least one coating layer containing or consisting of the enteric coating polymer.

Preferably, the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers; preferably the enteric coating polymer is selected from the group comprising or consisting of: hypromellose acetate succinate (HPMCAS), hypromellose phthalate, methacrylic acid-methyl methacrylate copolymers, methacrylic acid-ethyl acrylate copolymers, methyl acrylate-methyl methacrylate-methacrylic acid copolymers, polyvinyl acetate phthalate, cellulose acetate phthalate (CAP), shellac and a mixture of two or more of said enteric coating polymers.

More preferably, the enteric coating polymer is hypromellose acetate succinate and said hypromellose acetate succinate is selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

In some embodiments, the present invention refers to the oral formulation, wherein the at least one coating layer further comprises at least one buffering agent, plasticiser, and/or glidant.

Thus, said oral formulation adapted for selective delivery of a drug to the small intestine of a mammal comprises or consists of:
a plurality of particles in form of coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, or an enantiomer, a solvate, a hydrate or a pharmaceutically acceptable salt thereof;
      a core material and an enteric coating polymer,
      the core material comprises or consists of at least one buffering agent, plasticiser, and/or glidant,
      wherein each particle is coated with at least one coating layer containing or consisting of the enteric coating polymer.

Preferably, the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers; and

more preferably the enteric coating polymer is selected from the group comprising or consisting of: hypromellose acetate succinate (HPMCAS), hypromellose phthalate, methacrylic acid-methyl methacrylate copolymers, methacrylic acid-ethyl acrylate copolymers, methyl acrylate-methyl methacrylate-methacrylic acid copolymers, polyvinyl acetate phthalate, cellulose acetate phthalate (CAP), shellac and a mixture of two or more of said enteric coating polymers.

The hypromellose acetate succinate is preferably selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

Thus, said oral formulation adapted for selective delivery of a drug to the small intestine of a mammal comprises or consists of:
a plurality of particles in form of Thus, said oral formulation adapted for selective delivery of a drug to the small intestine of a mammal comprises or consists of:
   a plurality of particles in form of coated minitablets, each particle containing (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof;
   a core material, and the core material comprises one or more binder(s), disintegrants(s), glidant(s) and/or filler(s),
      wherein the binder is low-substituted hydroxypropylcellulose, hydroxypropylcellulose, or a mixture thereof;
      the disintegrant is croscarmellose sodium;
      the glidant is talc; silicon dioxide, or a mixture thereof;
      the pH-modifier is adipic acid; and/or
      the filler is mannitol; and
   an enteric coating polymer,
      wherein each particle is coated with at least one coating layer containing or consisting of the enteric coating polymer, at least one buffering agent, plasticiser, and/or glidant; preferably, the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

The hypromellose acetate succinate is preferably selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

Preferably, the buffering agent is ammonium hydrogen carbonate; the plasticiser is triethyl citrate; and/or the glidant is talc.

Thus, said oral formulation adapted for selective delivery of a drug to the small intestine of a mammal comprises or consists of:
a plurality of particles in form of coated minitablets, each particle containing (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, or an enantiomer, a solvate, a hydrate or a pharmaceutically acceptable salt thereof; a core material, and the core material comprises one or more binder(s), disintegrants(s), glidant(s) and/or filler(s),
   wherein the binder is low-substituted hydroxypropylcellulose, hydroxypropylcellulose, or a mixture thereof;
   the disintegrant is croscarmellose sodium;
   the glidant is talc, silicon dioxide, or a mixture thereof;
   the pH-modifier is adipic acid; and/or
   the filler is mannitol; and
an enteric coating polymer,
   wherein each particle is coated with at least one coating layer containing or consisting of the enteric coating polymer, at least one buffering agent, plasticiser, and/or glidant; preferably, the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

The hypromellose acetate succinate is preferably selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

Thus, said oral formulation adapted for selective delivery of a drug to the small intestine of a mammal comprises or consists of:
a plurality of particles in form of coated minitablets, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof;
   a core material, and the core material comprises one or more binder(s), disintegrants(s), glidant(s) pH-modifier(s) and/or filler(s),
      wherein the binder is low-substituted hydroxypropylcellulose or hydroxypropylcellulose;
      the disintegrant is croscarmellose sodium;
      the glidant is talc; silicon dioxide, and a mixture thereof;
      the pH-modifier is adipic acid; and/or
      the filler is mannitol; and
   an enteric coating polymer,
      wherein each particle is coated with at least one coating layer containing or consisting of the enteric coating polymer, at least one buffering agent, plasticiser, and/or glidant;
the buffering agent is ammonium hydrogen carbonate;
the plasticiser is triethyl citrate; and/or
the glidant is talc, silicon dioxide, or a mixture thereof,
   preferably, the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

The hypromellose acetate succinate is preferably selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

Preferably, the present invention is directed to the oral formulation as described above, wherein each particle contains
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof, and
50 wt % to 90 wt % of the core material comprising or consisting of the binder, the disintegrant, the glidant, the pH-modifier and/or the filler; and
1 wt % to 10 wt % of the enteric coating polymer.

In some embodiments, the present invention is directed to the oral formulation as described above, wherein each particle in form of a coated minitablet contains
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof, and
50 wt % to 90 wt % of the core material comprising or consisting of the binder, the disintegrant, the glidant, the pH-modifier and/or the filler; and
1 wt % to 10 wt % of the enteric coating polymer.

In some preferred embodiments, the present invention is directed to the oral formulation as described above, wherein each particle comprises or consists of:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
1 wt % to 10 wt % of HPMCAS;
15 wt % to 30 wt % of low-substituted hydroxypropylcellulose;
1 wt % to 10% of hydroxypropylcellulose;
15 wt % to 30 wt % of croscarmellose sodium;
15 wt % to 30 wt % of mannitol;
0.1 wt % to 3.0 wt % of triethyl citrate;
0 wt % to 1.0 wt % of ammonium hydrogen carbonate; and
5 wt % 10 wt % of talc, silicon dioxide, or a mixture thereof;
optionally, 5.0 wt % to 10.0 wt % of adipic acid.

Preferably, the present invention is directed to the oral formulation as described above, wherein each particle in form of a coated minitablet comprises or consists of:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
the core material comprising
   15 wt % to 30 wt % of low-substituted hydroxypropylcellulose;
   1 wt % to 10% of hydroxypropylcellulose;
   15 wt % to 30 wt % of croscarmellose sodium;
   15 wt % to 30 wt % of mannitol; and
   optionally, 5.0 wt % to 10.0 wt % of adipic acid;
the at least one coating layer comprises
   1 wt % to 10 wt % of HPMCAS;
   0 wt % to 1.0 wt % of ammonium hydrogen carbonate;
   0.1 wt % to 3.0 wt % of triethyl citrate; and
   5 wt % 10 wt % of talc, silicon dioxide, or a mixture thereof.

In some preferred embodiments, the present invention is directed to the oral formulation as described above, wherein each particle comprises or consists of:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
1 wt % to 10 wt % of Eudragit® L100/S100; preferably 1 wt % to 10% Eudragit®
L100 and 0 wt % to 5 wt % Eudragit® S100;
15 wt % to 30 wt % of low-substituted hydroxypropylcellulose;
1 wt % to 10% of hydroxypropylcellulose;
15 wt % to 30 wt % of croscarmellose sodium;
15 wt % to 30 wt % of mannitol;
0.1 wt % to 3.0 wt % of triethyl citrate;
3 wt % 10 wt % of talc, silicon dioxide, or a mixture thereof; and
optionally, 5.0 wt % to 10.0 wt % of adipic acid.

Preferably, the present invention is directed to the oral formulation as described above, wherein each particle in form of a coated minitablet comprises or consists of:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
the core material comprising
   20 wt % to 30 wt % of low-substituted hydroxypropylcellulose;
   1 wt % to 10% of hydroxypropylcellulose;
   20 wt % to 30 wt % of croscarmellose sodium;
   20 wt % to 30 wt % of mannitol;
   optionally, 5.0 wt % to 10.0 wt % of adipic acid;
the at least one coating layer comprises
   15 wt % to 25 wt % of Eudragit® L100/S100, preferably 1 wt % to 10% Eudragit® L100 and 0 wt % to 5 wt % Eudragit® S100;
   0.1 wt % to 3.0 wt % of triethyl citrate; and/or
   3 wt % 10 wt % of talc, silicon dioxide, or a mixture thereof.

More preferably, the present invention is directed to the oral formulation as described above, wherein each particle in form of a coated minitablet and each particle consists of:
8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
3 wt % to 8 wt % of HPMCAS; or 3 wt % to 8 wt % of Eudragit® L100/S100;
20 wt % to 26 wt % of low-substituted hydroxypropylcellulose;
2 wt % to 4 wt % of hydroxypropylcellulose;
20 wt % to 26 wt % of croscarmellose sodium
20 wt % to 26 wt % of mannitol;
0.1 to 1.0 wt % of triethyl citrate;
0.01 wt % to 1.0 wt % of ammonium hydrogen carbonate;
4 wt % to 8 wt % of talc; and
0.5 wt % to 1.5 wt % of silicon dioxide; and
optionally 5 wt % to 10 wt % of adipic acid.

More preferably, the oral formulation as described above, wherein each particle in form of a coated minitablet and each particle consists of:
8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
the core material comprising
   20 wt % to 26 wt % of low-substituted hydroxypropylcellulose;
   2 wt % to 4 wt % of hydroxypropylcellulose;
   20 wt % to 26 wt % of croscarmellose sodium
   20 wt % to 26 wt % of mannitol;
   optionally, 5 wt % to 10 wt % of adipic acid.
the at least one coating layer comprising
   3 wt % to 8 wt % of HPMCAS; or 3 wt % to 8 wt % of Eudragit® L100/S100;
   0.1 to 1.0 wt % of triethyl citrate;
   0.01 wt % to 1.0 wt % of ammonium hydrogen carbonate;
   4 wt % to 8 wt % of talc; and
   0.5 wt % to 1.5 wt % of silicon dioxide.

Still more preferably, the present invention is directed to the oral formulation as described above, wherein each particle in form of a coated minitablet and
each particle consists of:
   8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   3 wt % to 6 wt % of HPMCAS;
   20 wt % to 26 wt % of low-substituted hydroxypropylcellulose;
   2 wt % to 4 wt % of hydroxypropylcellulose;
   20 wt % to 26 wt % of croscarmellose sodium
   20 wt % to 26 wt % of mannitol;
   0.1 wt % to 1.0 wt % of triethyl citrate;
   0.01 wt % to 0.5 wt % of ammonium hydrogen carbonate;
   4 wt % to 8 wt % of talc;
   0.5 wt % to 1.5 wt % of silicon dioxide; and
   optionally, 6 wt % to 10 wt % of adipic acid;
or
each particle consists of:
   8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   3 wt % to 6 wt % of Eudragit® L100;
   0.1 wt to 1.5 wt % of Eudragit® S100;
   20 wt % to 26 wt % of low-substituted hydroxypropylcellulose;
   2 wt % to 4 wt % of hydroxypropylcellulose;
   20 wt % to 26 wt % of croscarmellose sodium
   20 wt % to 26 wt % of mannitol;
   0.1 wt % to 1.0 wt % of triethyl citrate;
   4 wt % to 8 wt % of talc;
   0.5 wt % to 1.5 wt % of silicon dioxide, and
   optionally, 6 wt % to 10 wt % of adipic acid.

Still more preferably, the present invention is directed to the oral formulation as described above, wherein each particle in form of a coated minitablet and
each particle consists of:
   8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   the core material comprising
      20 wt % to 26 wt % of low-substituted hydroxypropylcellulose;
      2 wt % to 4 wt % of hydroxypropylcellulose;
      20 wt % to 26 wt % of croscarmellose sodium
      20 wt % to 26 wt % of mannitol;
      optionally, 6 wt % to 10 wt % of adipic acid; and
   the at least one coating layer comprising
      3 wt % to 6 wt % of HPMCAS;
      0.1 wt % to 1.0 wt % of triethyl citrate;
      0.01 wt % to 0.5 wt % of ammonium hydrogen carbonate;
      4 wt % to 8 wt % of talc;
      0.5 wt % to 1.5 wt % of silicon dioxide;
or
each particle consists of:
   8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   the core material comprising
      20 wt % to 26 wt % of low-substituted hydroxypropylcellulose;
      2 wt % to 4 wt % of hydroxypropylcellulose;
      20 wt % to 26 wt % of croscarmellose sodium;
      20 wt % to 26 wt % of mannitol;
      optionally, 6 wt % to 10 wt % of adipic acid; and
   the at least one coating layer comprising
      3 wt % to 6 wt % of Eudragit® L100;
      0.1 wt to 1.5 wt % of Eudragit® S100;
      0.1 wt % to 1.0 wt % of triethyl citrate;
      4 wt % to 8 wt % of talc;
      0.5 wt % to 1.5 wt % of silicon dioxide.

Most preferably, the present invention is directed to the oral formulation as described above, wherein each particle in form of a coated minitablet and each particle consists of:
9.22 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
4.06 wt % of HPMCAS;
22.12 wt % of low-substituted hydroxypropylcellulose;
2.77 wt % of hydroxypropylcellulose;
23.04 wt % of croscarmellose sodium
23.04 wt % of mannitol;
0.64 wt % of triethyl citrate;
8.3 wt % of adipic acid;
0.07 wt % of ammonium hydrogen carbonate;
5.83 wt % of talc; and
0.92 wt % of silicon dioxide;
   or
   each particle consists of:
10.05 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
4.42 wt % of HPMCAS;
24.12 wt % of low-substituted hydroxypropylcellulose;
3.02 wt % of hydroxypropylcellulose;
25.13 wt % of croscarmellose sodium
25.13 wt % of mannitol;
0.69 wt % of triethyl citrate;
0.08 wt % of ammonium hydrogen carbonate;
6.35 wt % of talc; and
1.01 wt % of silicon dioxide;
   or
   each particle consists of:
9.22 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
3.74 wt % of Eudragit® L100;
0.66 wt % of Eudragit® S100;
22.12 wt % of low-substituted hydroxypropylcellulose;
2.76 wt % of hydroxypropylcellulose;
23.04 wt % of croscarmellose sodium
23.04 wt % of mannitol;
0.43 wt % of triethyl citrate;
8.29 wt % of adipic acid;
5.76 wt % of talc; and
0.92 wt % of silicon dioxide;
   or
   each particle consists of:
10.05 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
4.08 wt % of Eudragit® L100;
0.72 wt % of Eudragit® S100;
24.12 wt % of low-substituted hydroxypropylcellulose;
3.02 wt % of hydroxypropylcellulose;
25.13 wt % of croscarmellose sodium
25.13 wt % of mannitol;
0.47 wt % of triethyl citrate;
6.28 wt % of talc; and
1.01 wt % of silicon dioxide.

In another aspect, the oral formulation of the present invention contains a core material in form of a core pellet consisting of: tartaric acid, lactose, sugar, maize starch, starch hydrolysates, silica, or microcrystalline cellulose, preferred, microcrystalline cellulose.

Hence, in some embodiments, the invention refers to the oral formulation adapted for selective delivery of a drug to the small intestine of a mammal comprising or consisting of:
a plurality of particles in form of coated granules, coated pellets, coated beads, or coated minicapsules, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   a core material, the core material is in form of a core pellet consisting of: tartaric acid, lactose, sugar, maize starch, starch hydrolysates, silica, or microcrystalline cellulose; and
   an enteric coating polymer,
      wherein each particle is coated with at least one coating layer containing or consisting of the enteric coating polymer.

Preferably, the core material is in form of a core pellet consisting of microcrystalline cellulose.

Preferably, the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

The hypromellose acetate succinate is preferably selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

In some embodiments, the present invention is directed to the oral formulation, wherein in case the particle is in form of a coated granule, a coated pellet, a coated bead, or a coated minicapsule, the core material is in form of the core pellet consisting of: tartaric acid, lactose, sugar, maize starch, starch hydrolysates, silica, or microcrystalline cellulose, preferably microcrystalline.

In some embodiments, the present invention is directed to the oral formulation, wherein in case the particle is in form of a coated granule, a coated pellet, a coated bead, or a coated minicapsule, the core material is in form of the core pellet consisting of: tartaric acid, lactose, sugar, maize starch, starch hydrolysates, silica, or microcrystalline cellulose, preferably microcrystalline; and

wherein the core pellet is coated with (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof, together with at least one binder(s), colorant(s), glidant(s), surfactant(s) and/or filler(s).

In some embodiments, in the oral formulation as described above, the enteric coating polymer is contained in or forms the outer layer of the coating enclosing the core material. Thus, the present invention is directed to the oral formulation adapted for selective delivery of a drug to the small intestine of a mammal comprising or consisting of:
a plurality of particles in form of coated granules, coated pellets, coated beads, or coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   a core material, the core material is in form of a core pellet selected from the group consisting of: tartaric acid, lactose, sugar, maize starch, starch hydrolysates, silica, and microcrystalline cellulose; preferably microcrystalline cellulose and
an enteric coating polymer,
   wherein each particle is coated with at least one coating layer containing or consisting of the enteric coating polymer, and the enteric coating polymer is contained in or forms the outer layer of the coating enclosing the core material.

The oral formulation containing the core material in form of a core pellet as described herein, may further comprise at least one binder, colorant, glidant, surfactant and/or filler.

Thus, in some embodiments, the present invention is directed to an oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
   a core material, the core material is in form of a core pellet consisting of: tartaric acid, lactose, sugar, maize starch, starch hydrolysates, silica, or microcrystalline cellulose;
   and an enteric coating polymer,
wherein each particle is coated with at least one coating layer containing or consisting of the enteric coating polymer.

Preferably, the core material is in form of a core pellet consisting of microcrystalline cellulose, more preferably Cellets.

Cellets are highly spherical core pellets consisting of 100% microcrystalline cellulose. The size of the microcrystalline cellulose core pellets can range from 100 to 200 μm (Cellets 100), 200 to 355 μm (Cellets 200), 350 to 500 μm (Cellets 350), 500 to 710 μm (Cellets 500), 700 to 1000 μm (Cellets 700) and from 1000 to 14000 μm (Cellets 1000).

More preferably, the particle size of the microcrystalline cellulose core pellets range from 200 μm to 710 μm (Cellets 200, 350, 500), still more preferably, 350 μm to 710 μm (Cellet 350, 500), and most preferably 500 μm to 710 μm (Cellet 500).

In some embodiments, the present invention refers to the oral formulation, wherein the core material is in form of a core pellet as described above, the size of the core pellet is in a range from 100 μm to 14000 μm.

In some embodiments, the present invention refers to the oral formulation, wherein the core material is in form of a core pellet as described above, the core pellet consists of microcrystalline cellulose and the size of the core pellet is in a range from 500 μm to 710 μm (Cellet 500).

As used herein, the colorant is selected from titanium dioxide, iron(III)oxide, iron(II,III) oxide, hydrated ferric oxide, lactose monohydrate, carnauba wax, and a mixture thereof.

As used herein, the surfactant is preferably anionic surfactants contain anionic functional groups such as sulfate, sulfonate, phosphate, and carboxylate.

Preferably anionic surfactants include but not limited to ammonium lauryl sulfate, sodium dodecyl sulfate (sodium lauryl sulfate), sodium laureth sulfate, and sodium myreth sulfate, preferably, sodium dodecyl sulfate.

Preferably, the oral formulation, wherein the core material is in form of a core pellet as described above, and said oral formulation further comprises at least one binder(s), colorant(s), glidant(s), surfactant(s) and/or filler(s),

wherein
the binder is polyvinylpyrrolidone (povidone);
the surfactant is sodium dodecyl sulfate; and/or
the filler is lactose monohydrate.

Thus, the present invention is directed to the oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
      wherein
      the binder is polyvinylpyrrolidone (povidone);
      the surfactant is sodium dodecyl sulfate; and/or
      the filler is lactose monohydrate;
   a core material, the core material is in form of a core pellet consisting of: tartaric acid, lactose, sugar, maize starch, starch hydrolysates, silica, or microcrystalline cellulose;
   and an enteric coating polymer,
wherein each particle is coated with at least one coating layer containing or consisting of the enteric coating polymer.

Povidone K25, povidone K30 or povidone K90 is commercially available and preferably, povidone K25 is used as a binder. The approximate average molecular weight of povidone K25 is 30,000 g/mol (Da) between 28,000 g/mol (Da) to 34,000 g/mol (Da).

In the oral formulation containing the core material in form of a core pellet as described herein, the layer containing the enteric coating polymer may further comprise at least one buffering agent, plasticiser, and/or glidant.

Thus, in some embodiment, the present invention is directed to the oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
   a core material, the core material is in form of a core pellet consisting of: tartaric acid, lactose, sugar, maize starch, starch hydrolysates, silica, or microcrystalline cellulose;
   and an enteric coating polymer,
      wherein each particle is coated with at least one coating layer containing or consisting of the enteric coating polymer, and the layer containing the enteric coating polymer further comprises at least one buffering agent, plasticiser, and/or glidant.

Preferably, the buffering agent is ammonium hydrogen carbonate;
the plasticiser is triethyl citrate; and/or
the glidant is talc.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
      wherein
      the binder is polyvinylpyrrolidone (povidone);
      the surfactant is sodium dodecyl sulfate; and/or
      the filler is lactose monohydrate;
   a core material, the core material is in form of a core pellet consisting of: tartaric acid, lactose, sugar, maize starch, starch hydrolysates, silica, or microcrystalline cellulose;
   and an enteric coating polymer,
wherein each particle is coated with at least one coating layer containing or consisting of the enteric coating polymer and the layer containing the enteric coating polymer further comprises at least one buffering agent, plasticiser, and/or glidant.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
      wherein
      the binder is polyvinylpyrrolidone (povidone);
      the colorant is selected from titanium dioxide, iron(III)oxide, iron(II,III) oxide, hydrated ferric oxide, lactose monohydrate, carnauba wax, and a mixture thereof;
      the surfactant is sodium dodecyl sulfate; and/or
      the filler is lactose monohydrate;
   a core material, the core material is in form of a core pellet consisting of: tartaric acid, lactose, sugar, maize starch, starch hydroylsates, silica, or microcrystalline cellulose;
   and an enteric coating polymer,
wherein each particle is coated with at least one coating layer containing or consisting of the enteric coating polymer and the layer containing the enteric coating polymer further comprises at least one buffering agent, plasticiser, and/or glidant, wherein
   the buffering agent is ammonium hydrogen carbonate;
   the plasticiser is triethyl citrate; and/or
   the glidant is talc.

Preferably, the oral formulation, wherein the core material is in form of a core pellet as described above, comprises the binder(s), the colorant(s), the surfactant (s), the filler(s), the buffering agent(s), the plasticiser(s), and/or the glidant(s), and
the binder is povidone K25;
the colorant is selected from titanium dioxide, iron(III)oxide, iron(II,III) oxide, hydrated ferric oxide, lactose monohydrate, carnauba wax, and a mixture thereof; the filler is lactose monohydrate;
the buffering agent is ammonium hydrogen carbonate;
the plasticiser is triethyl citrate; and/or
the glidant is talc.

Thus, in some embodiments, the oral formulation adapted for selective delivery of a drug to the small intestine of a mammal comprises or consists of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
      wherein
      the binder is polyvinylpyrrolidone (povidone);
      the colorant is selected from titanium dioxide, iron(III)oxide, iron(II,III) oxide, hydrated ferric oxide, lactose monohydrate, carnauba wax, and a mixture thereof;
      the surfactant is sodium dodecyl sulfate; and/or
      the filler is lactose monohydrate;
   a core material, the core material is in form of a core pellet consisting of: tartaric acid, lactose, sugar, maize starch, starch hydrolyates, silica, or microcrystalline cellulose;
   and an enteric coating polymer,
wherein each particle is coated with at least one coating layer containing or consisting of the enteric coating polymer and the layer containing the enteric coating polymer further comprises at least one buffering agent, plasticiser, and/or glidant, wherein
   the buffering agent is ammonium hydrogen carbonate;
   the plasticiser is triethyl citrate; and/or
   the glidant is talc.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   a core material, the core material is in form of a core pellet consisting of: tartaric acid, lactose, sugar, maize starch, starch hydrolyates, silica, or microcrystalline cellulose; preferably microcrystalline cellulose;
   at least one layer containing or consisting of
      (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
      and
   at least one coating layer containing or consisting of the enteric coating polymer,
   at least one buffering agent, plasticiser, and/or glidant,
   wherein each particle is coated with the at least one coating layer.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   a core material, the core material is in form of a core pellet consisting of: tartaric acid, lactose, sugar, maize starch, starch hydrolyates, silica, or microcrystalline cellulose; preferably microcrystalline cellulose:
   at least one layer containing or consisting of
      (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
         wherein
         the binder is polyvinylpyrrolidone (povidone);
         the colorant is selected from titanium dioxide, iron(III)oxide, iron(II,III) oxide, hydrated ferric oxide, lactose monohydrate, carnauba wax, and a mixture thereof;
         the surfactant is sodium dodecyl sulfate; and/or
         the filler is lactose monohydrate;
   wherein the core material is coated with said at least one layer;
   at least one coating layer containing or consisting of the enteric coating polymer,
   at least one buffering agent, plasticiser, and/or glidant,
      wherein
      the buffering agent is ammonium hydrogen carbonate;
      the plasticiser is triethyl citrate; and/or
      the glidant is talc;
   wherein each particle is coated with the at least one coating layer.

In another aspect, the present invention refers to the oral formulation as mentioned above, wherein each particle is coated further with a coating layer comprising or consisting of a sustained-release polymer.

Hence, in some embodiments, the invention refers to the oral formulation adapted for selective delivery of a drug to the small intestine of a mammal comprising or consisting of:
a plurality of particles in form of coated granules, coated pellets, coated beads, or coated minicapsules,
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   a core material, the core material is in form of a core pellet consisting of: tartaric acid, lactose, sugar, maize starch, starch hydroxylates, silica, or microcrystalline cellulose; and
   an enteric coating polymer,
      wherein each particle is coated with at least one coating layer containing or consisting of the enteric coating polymer and coated further with a coating layer comprising or consisting of a sustained-release polymer.

The sustained-release polymer may be firstly coated under the coating layer containing or consisting of the enteric coating polymer; or the sustained-release polymer may be later coated on the coating layer containing or consisting of the enteric coating polymer.

Preferably, the sustained-release polymer is selected from hypromellose (HPMC), ethylcellulose, ammonio methacrylate co-polymers such as Eudragit® RL100, Eudragit® RS100, Eudragit® RL12.5, Eudragit® RS12.5, and a mixture thereof.

In case the sustained-release polymer is ethylcellulose, optionally polyethylene glycol (PEG) is further comprised as plasticizer, preferably Macrogol® 6000.

Hypromellose (HPMC) may be firstly coated under the coating layer containing or consisting of the enteric coating polymer. Ethylcellulose, Eudragit® RL100, Eudragit® RS100, Eudragit® RL12.5, Eudragit® RS12.5 may be later coated on the coating layer containing or consisting of the enteric coating polymer.

In case the sustained-release polymer is ethylcellulose, optionally polyethylene glycol (PEG) is further comprised as plasticizer, preferably Macrogol® 6000.

Ammonio methacrylate co-polymers such as Eudragit® RL100 and Eudragit® RS100 may be suitable for use in the modified release formulations of the present invention.

These polymers are insoluble in pure water, dilute acids, buffer solutions, or digestive fluids over the entire physiological pH range. The polymers swell in water and digestive fluids independently of pH. In the swollen state, they are then permeable to water and dissolved active agents. The permeability of the polymers depends on the ratio of ethylacrylate (EA), methyl methacrylate (MMA), and trimethylammonioethyl methacrylate chloride (TAMCI) groups in the polymer. Those polymers having EA:MMA:TAMC1 ratios of 1:2:0.2 (Eudragit® RL100) are more permeable than those with ratios of 1:2:0.1 (Eudragit® RS100). Polymers of Eudragit® RL100 are insoluble polymers of high permeability. Polymers of Eudragit® RS100 are insoluble films of low permeability.

The ammonio methacrylate co-polymers can be combined in any desired ratio, and the ratio can be modified to modify the rate of drug release. For example, a ratio of Eudragit® RS100:Eudragit® RL100 of 90:10 can be used. Alternatively, the ratio of Eudragit® RS100:Eudragit® RL100 can be about 100:0 to about 80:20, or about 100:0 to about 90:10, or any ratio in between. In such formulations, the less permeable polymer Eudragit® RS100 would generally comprise the majority of the polymeric material with the more permeable RL, permitting and creating gaps through which solutes can enter the core and dissolved pharmaceutical active ingredients can escape in a controlled manner.

The ammonio methacrylate co-polymers can be combined with the methacrylic acid copolymers within the polymeric material in order to achieve the desired delay in the release of the drug. Ratios of amino methacrylate co-polymer (e.g., Eudragit® RS100) to methacrylic acid co-polymer in the range of about 99:1 to about 20:80 can be used. The two types of polymers can also be combined into the same polymeric material, or provided as separate coats that are applied to the core.

Eudragit® RL12.5 and Eudragit® RL100 are ammonio methacrylate copolymers (Type A)=Poly[ethyl propenoate-co-methyl 2-methylprop-2-enoate-co-N,N,N-trimethyl-2-[(2-methylprop-2-enoyl)oxy]ethan-1-aminium chloride] with a mean relative molecular mass of about 150 000. The ratio of ethyl acrylate groups to methyl methacrylate groups to ammonio methacrylate groups is about 1:2:0.2.

Eudragit® RS12.5 and Eudragit® RS100 are ammonio methacrylate copolymers (Type B)=Poly[ethyl propenoate-co-methyl 2-methylprop-2-enoate-co-N,N,N-trimethyl-2-[(2-methylprop-2-enoyl)oxy]ethan-1-aminium chloride] with a mean relative molecular mass of about 150 000. The ratio of ethyl acrylate groups to methyl methacrylate groups to ammonio methacrylate groups is about 1:2:0.1.

In some embodiments, the present invention is directed to the oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
   a core material, the core material is in form of a core pellet consisting of: tartaric acid, lactose, sugar, maize starch, starch hydrolysates, silica, or microcrystalline cellulose;
   and an enteric coating polymer,
wherein each particle is coated with at least one coating layer containing or consisting of the enteric coating polymer and coated further with a coating layer comprising or consisting of a sustained-release polymer.

In some embodiments, the present invention is directed to the oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
   a core material, the core material is in form of a core pellet consisting of: tartaric acid, lactose, sugar, maize starch, starch hydrolysates, silica, or microcrystalline cellulose;
   and an enteric coating polymer,
wherein each particle is firstly coated with a coating layer comprising or consisting of a sustained-release polymer, and subsequently further coated with at least one coating layer containing or consisting of the enteric coating polymer.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   a core material, the core material is in form of a core pellet consisting of: tartaric acid, lactose, sugar, maize starch, starch hydrolyates, silica, or microcrystalline cellulose; preferably microcrystalline cellulose:
   at least one layer containing or consisting of
      (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
   a coating layer comprising or consisting of a sustained-release polymer; and
   at least one coating layer containing or consisting of the enteric coating polymer,
   at least one buffering agent, plasticiser, and/or glidant,
   wherein each particle is coated with the at least one coating layer.
wherein each core material is firstly coated with the coating layer comprising or consisting of a sustained-release polymer, and later further coated with the at least one coating layer containing or consisting of the enteric coating polymer.

In some embodiments, the represent invention is directed to the oral formulation adapted for selective delivery of the drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
      wherein
      the binder is polyvinylpyrrolidone (povidone);
      the surfactant is sodium dodecyl sulfate; and/or
      the filler is lactose monohydrate;
   a core material, the core material is in form of a core pellet consisting of: tartaric acid, lactose, sugar, maize starch, starch hydroxylates, silica, or microcrystalline cellulose;
   and an enteric coating polymer,
wherein each particle is coated with at least one coating layer containing or consisting of the enteric coating polymer and coated further with a coating layer comprising or consisting of a sustained-release polymer.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   a core material, the core material is in form of a core pellet consisting of: tartaric acid, lactose, sugar, maize starch, starch hydrolyates, silica, or microcrystalline cellulose; preferably microcrystalline cellulose;
   at least one layer containing or consisting of
      (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
         wherein
         the binder is polyvinylpyrrolidone (povidone);
         the surfactant is sodium dodecyl sulfate; and/or
         the filler is lactose monohydrate;
   a coating layer comprising or consisting of a sustained-release polymer; and
   at least one coating layer containing or consisting of the enteric coating polymer,
   at least one buffering agent, plasticiser, and/or glidant,
   wherein each particle is coated with the at least one coating layer,
wherein each particle is coated with at least one coating layer containing or consisting of the enteric coating polymer and coated further with a coating layer comprising or consisting of a sustained-release polymer.
Preferred are these oral formulations, wherein each core material is firstly coated with the coating layer comprising or consisting of a sustained-release polymer, and subsequently further coated with the at least one coating layer containing or consisting of the enteric coating polymer, or
wherein each core material is firstly coated with the at least one coating layer containing or consisting of the enteric coating polymer; and subsequently coated with the coating layer comprising or consisting of a sustained-release polymer.

In some embodiments, the present invention is directed to the oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
   a core material, the core material is in form of a core pellet consisting of: tartaric acid, lactose, sugar, maize starch, starch hydrolysates, silica, or microcrystalline cellulose, preferably microcrystalline cellulose;
   and an enteric coating polymer,
wherein each particle is coated with at least one coating layer containing or consisting of the enteric coating polymer and the layer containing the enteric coating polymer further comprises at least one buffering agent, plasticiser, and/or glidant and each particle is coated further with a coating layer comprising or consisting of a sustained-release polymer.

In some embodiments, the oral formulation adapted for selective delivery of the drug to the small intestine of a mammal comprises or consists of:
a plurality of particles in form of coated granules, coated pellets, coated beads, coated minicapsules;
each particle containing
   a core material, the core material is in form of a core pellet consisting of: tartaric acid, lactose, sugar, maize starch, starch hydrolyates, silica, or microcrystalline cellulose; preferably microcrystalline cellulose;
   at least one layer containing or consisting of
      (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
   at least one coating layer containing or consisting of the enteric coating polymer,
   at least one buffering agent, plasticiser, and/or glidant,
   a coating layer comprising or consisting of a sustained-release polymer;
      wherein each particle is coated with the at least one coating layer containing or consisting of the enteric coating polymer; and coated further with the coating layer comprising or consisting of a sustained-release polymer.
Preferred are these oral formulations, wherein each core material is firstly coated with the coating layer comprising or consisting of a sustained-release polymer, and subsequently further coated with the at least one coating layer containing or consisting of the enteric coating polymer; or
wherein each core material is firstly coated with the at least one coating layer containing or consisting of the enteric coating polymer; and subsequently coated with the coating layer comprising or consisting of a sustained-release polymer.

In some embodiments, the present invention is directed to the oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
      wherein
      the binder is polyvinylpyrrolidone (povidone);
      the colorant is selected from titanium dioxide, iron(III)oxide, iron(II,III) oxide, hydrated ferric oxide, lactose monohydrate, carnauba wax, and a mixture thereof;
      the surfactant is sodium dodecyl sulfate; and/or
      the filler is lactose monohydrate;
   a core material, the core material is in form of a core pellet consisting of: tartaric acid, lactose, sugar, maize starch, starch hydrolysates, silica, or microcrystalline cellulose;
   and an enteric coating polymer,
wherein each particle is coated with at least one coating layer containing or consisting of the enteric coating polymer and the layer containing the enteric coating polymer further comprises at least one buffering agent, plasticiser, and/or glidant and each particle is coated further with a coating layer comprising or consisting of a sustained-release polymer.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   a core material, the core material is in form of a core pellet consisting of: tartaric acid, lactose, sugar, maize starch, starch hydrolyates, silica, or microcrystalline cellulose; preferably microcrystalline cellulose;
   at least one layer containing or consisting of
      (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
         wherein
         the binder is polyvinylpyrrolidone (povidone);
         the colorant is selected from titanium dioxide, iron(III)oxide, iron(II,III) oxide, hydrated ferric oxide, lactose monohydrate, carnauba wax, and a mixture thereof;
         the surfactant is sodium dodecyl sulfate; and/or
         the filler is lactose monohydrate;
   at least one coating layer containing or consisting of the enteric coating polymer,
   at least one buffering agent, plasticiser, and/or glidant,
   a coating layer comprising or consisting of a sustained-release polymer;
      wherein each particle is coated with the at least one coating layer containing or consisting of the enteric coating polymer; and coated further with the coating layer comprising or consisting of a sustained-release polymer.

Preferred are these oral formulations, wherein each core material is firstly coated with the coating layer comprising or consisting of a sustained-release polymer, and subsequently further coated with the at least one coating layer containing or consisting of the enteric coating polymer; or

wherein each core material is firstly coated with the at least one coating layer containing or consisting of the enteric coating polymer; and subsequently coated with the coating layer comprising or consisting of a sustained-release polymer.

In some embodiments, the present invention is directed to the oral formulation adapted for selective delivery of a drug to the small intestine of a mammal comprising or consisting of:
a plurality of particles in form of coated granules, coated pellets, coated beads, coated minicapsules;
each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   together with at least one binder(s), colorant(s), glidant(s), surfactant(s) and/or filler(s);
      wherein
      the binder is polyvinylpyrrolidone (povidone);
      the colorant is selected from titanium dioxide, iron(III)oxide, iron(II,III) oxide, hydrated ferric oxide, lactose monohydrate, carnauba wax, and a mixture thereof;
      the surfactant is sodium dodecyl sulfate; and/or
      the filler is lactose monohydrate;
   a core material, the core material is in form of a core pellet consisting of: tartaric acid, lactose, sugar, maize starch, starch hydrolysates, silica, or microcrystalline cellulose;
   and an enteric coating polymer,
wherein each particle is coated with at least one coating layer containing or consisting of the enteric coating polymer and the layer containing the enteric coating polymer further comprises at least one buffering agent, plasticiser, and/or glidant;
wherein
   the buffering agent is ammonium hydrogen carbonate;
   the plasticiser is triethyl citrate; and/or
   the glidant is talc;
and
each particle is coated further with a coating layer comprising or consisting of a sustained-release polymer; preferably, the sustained-release polymer is selected from hypromellose (HPMC), ethylcellulose, ammonio methacrylate co-polymers such as Eudragit® RL100, Eudragit® RS100, Eudragit® RL12.5, Eudragit® RS12.5, and a mixture thereof.
In case the sustained-release polymer is ethylcellulose, optionally polyethylene glycol (PEG) is further comprised as plasticizer, preferably Macrogol® 6000.

In some embodiments, the oral formulation adapted for selective delivery of a drug to the small intestine of a mammal comprises or consists of:
a plurality of particles in form of coated granules, coated pellets, coated beads, coated minicapsules;
each particle containing
   a core material, the core material is in form of a core pellet consisting of: tartaric acid, lactose, sugar, maize starch, starch hydrolyates, silica, or microcrystalline cellulose; preferably microcrystalline cellulose;
   at least one layer containing or consisting of
      (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, together with at least one binder(s), colorant(s), glidant(s), surfactant(s) and/or filler(s);
         wherein
         the binder is polyvinylpyrrolidone (povidone);
         the colorant is selected from titanium dioxide, iron(III)oxide, iron(II,III) oxide, hydrated ferric oxide, lactose monohydrate, carnauba wax, and a mixture thereof;
         the surfactant is sodium dodecyl sulfate; and/or
         the filler is lactose monohydrate;
   at least one coating layer containing or consisting of the enteric coating polymer,
   at least one buffering agent, plasticiser, and/or glidant,
      wherein
      the buffering agent is ammonium hydrogen carbonate;
      the plasticiser is triethyl citrate; and/or
      the glidant is talc;
   a coating layer comprising or consisting of a sustained-release polymer.
      wherein each particle is coated with the at least one coating layer containing or consisting of the enteric coating polymer; and coated further with the coating layer comprising or consisting of a sustained-release polymer.

Preferred are these oral formulations, wherein each core material is firstly coated with the coating layer comprising or consisting of a sustained-release polymer, and subsequently further coated with the at least one coating layer containing or consisting of the enteric coating polymer; or

wherein each core material is firstly coated with the at least one coating layer containing or consisting of the enteric coating polymer; and subsequently coated with the coating layer comprising or consisting of a sustained-release polymer.

Preferred are oral formulations as described herein consisting of: a plurality of particles in form of coated granules, coated pellets, coated beads, coated minicapsules; wherein

each particle comprises:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
30 wt % to 55 wt % of the core material; the core material consisting of tartaric acid, lactose, sugar, maize starch, starch hydrolysates, silica, or microcrystalline cellulose; preferably microcrystalline cellulose; and
15 wt % to 30 wt % of the enteric coating polymer.

In some embodiments, the present invention is directed to the oral formulation adapted for selective delivery of Compound 1 to the small intestine of a mammal comprising or consisting of:
a plurality of particles in form of coated granules, coated pellets, coated beads, coated minicapsules;
each particle containing
   5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof;
   15 wt % to 30 wt % of the enteric coating polymer;
   30 wt % to 55 wt % of the core material, the core material consisting of tartaric acid, lactose, sugar, maize starch, starch hydrolysates, silica, or microcrystalline cellulose; and preferably of microcrystalline cellulose;
wherein each particle is coated further with a coating layer comprising or consisting of a sustained-release polymer.

Preferred are oral formulations as described herein consisting of: a plurality of particles in form of coated granules, coated pellets, coated beads, coated minicapsules; wherein

each particle comprises:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, and
30 wt % to 55 wt % of the core material; and
15 wt % to 30 wt % of the enteric coating polymer and
0.1 wt to 2.0 wt % of the sustained-release polymer.

Preferred are also oral formulations as described herein consisting of: a plurality of particles in form of coated granules, coated pellets, coated beads, coated minicapsules; wherein the core material is in form of a core pellet consisting of microcrystalline cellulose;

each particle comprises:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
30 wt % to 45 wt % of microcrystalline cellulose core;
15 wt % to 25 wt % of HPMCAS;
10 wt % 20 wt % of lactose monohydrate;
10 wt % 15 wt % of talc;
1.0 wt % to 3.0 wt % of triethyl citrate,
0.1 wt % to 1.0 wt % of ammonium hydrogen carbonate; and
optionally, 0.1 wt % to 2.0 wt % of sodium dodecylsulfate; and/or
0.1 wt to 2.0 wt % of HPMC.

Preferred are oral formulations as described herein consisting of: a plurality of particles in form of coated granules, coated pellets, coated beads, coated minicapsules; wherein the core material is in form of a core pellet consisting of microcrystalline cellulose; each particle comprises:
30 wt % to 45 wt % of microcrystalline cellulose core as the core material;
at least one layer containing or consisting of
   5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   30 wt % to 45 wt % of microcrystalline cellulose core;
   10 wt % 20 wt % of lactose monohydrate;
   optionally, 0.1 wt % to 2.0 wt % of sodium dodecylsulfate;
at least one coating layer containing or consisting of
   15 wt % to 25 wt % of HPMCAS;
   10 wt % 15 wt % of talc;
   1.0 wt % to 3.0 wt % of triethyl citrate, and
   0.1 wt % to 1.0 wt % of ammonium hydrogen carbonate;
and/or
a coating layer comprising or consisting of
   0.1 wt to 2.0 wt % of HPMC as a sustained-release polymer.
      wherein each particle is coated with the at least one coating layer containing or consisting of HPMCAS; and coated further with the coating layer comprising or consisting of HPMC.

Preferred is when each core material is firstly coated with the coating layer comprising or consisting of HPMC and subsequently further coated with the at least one coating layer containing or consisting of HPMCAS.

Preferred are further oral formulations as described herein consisting of: a plurality of particles in form of coated granules, coated pellets, coated beads, coated minicapsules; wherein the core material is in form of a core pellet consisting of microcrystalline cellulose;

each particle comprises:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
30 wt % to 45 wt % of microcrystalline cellulose core
15 wt % to 30 wt % of Eudragit® L100 and 0 wt % to 5 wt % of Eudragit® S100;
10 wt % to 20 wt % of lactose monohydrate;
10 wt % to 15 wt % of talc;
0.5 wt % to 2.0 wt % of povidone K25;
1.0 wt % to 3.0 wt % of triethyl citrate; and/or
optionally, 0.1 wt % to 2.0 wt % of ethylcellulose and PEG; preferably PEG is Macrogol® 6000.

Preferred is when each core material is firstly coated with the coating layer comprising or consisting of Eudragit® L100 and Eudragit® S100, and subsequently further coated with the at least one coating layer containing or consisting of ethylcellulose and PEG.

Preferred are still oral formulations as described herein consisting of: a plurality of particles in form of coated granules, coated pellets, coated beads, coated minicapsules; wherein the core material is in form of a core pellet consisting of microcrystalline cellulose;
each particle comprises:
   30 wt % to 45 wt % of microcrystalline cellulose core as the core material; at least one layer containing or consisting of
      5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
      10 wt % to 20 wt % of lactose monohydrate;
      0.5 wt % to 2.0 wt % of povidone K25;
   at least one coating layer containing or consisting of
      15 wt % to 30 wt % of Eudragit® L100 and
      0 wt % to 5 wt % of Eudragit® S100;
      10 wt % to 15 wt % of talc;
      1.0 wt % to 3.0 wt % of triethyl citrate; and/or
optionally,
a coating layer comprising or consisting of 0.1 wt % to 2.0 wt % of ethylcellulose and PEG; preferably PEG is Macrogol® 6000.

Preferred is when each core material is firstly coated with the coating layer comprising or consisting of Eudragit® L100 and Eudragit® S100, and subsequently further coated with the at least one coating layer containing or consisting of ethylcellulose and PEG, preferably PEG is Macrogol® 6000.

Preferred are still oral formulations as described herein consisting of: a plurality of particles in form of coated granules, coated pellets, coated beads, coated minicapsules; wherein the core material is in form of a core pellet consisting of microcrystalline cellulose;
each said particle consists of:
   7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   37 wt % to 41 wt % of microcrystalline cellulose core (Cellets 500);
   15 wt % to 19 wt % of HPMCAS;
   14 wt % to 18 wt % of lactose monohydrate;
   11 wt % to 15 wt % of talc;
   0.5 wt % to 2.0 wt % of povidone K25;
   1.0 wt % to 3.0 wt % of triethyl citrate, and
   0.1 wt % to 1.0 wt % of ammonium hydrogen carbonate;
or
said particle consists of:
   7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   37 wt % to 41 wt % of microcrystalline cellulose core (Cellets 500);
   15 wt % to 19 wt % of HPMCAS;
   12 wt % to 16 wt % of lactose monohydrate;
   11 wt % to 15 wt % of talc;
   0.5 wt % to 2.0 wt % of povidone K25;
   1.0 wt % to 3.0 wt % of triethyl citrate:
   0.5 wt % to 2.0 wt % of sodium dodecylsulfate; and
   0.1 wt % to 1.0 wt % of ammonium hydrogen carbonate;
or
said particle consists of:
   7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   37 wt % to 41 wt % of microcrystalline cellulose core (Cellets 500);
   15 wt % to 19 wt % of HPMCAS;
   13 wt % to 17 wt % of lactose monohydrate;
   11 wt % to 15 wt % of talc;
   0.5 wt % to 2.0 wt % of povidone K25;
   1.0 wt % to 3.0 wt % of triethyl citrate:
   0.1 wt % to 1.0 wt % of ammonium hydrogen carbonate; and
   0.5 wt % to 2.0 wt % of HPMC;
or
said particle consists of:
   7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   33 wt % to 37 wt % of microcrystalline cellulose core (Cellets 500);
   20 wt % to 24 wt % of Eudragit® L100 and 3 wt % to 5 wt % of Eudragit® S100;
   12 wt % to 16 wt % of lactose monohydrate;
   12 wt % to 15 wt % of talc; and
   1.0 wt % to 3.0 wt % of triethyl citrate;
or
said particle consists of:
   7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   35 wt % to 39 wt % of microcrystalline cellulose core (Cellets 200);
   23 wt % to 27 wt % of Eudragit® L100;
   10 wt % to 14 wt % of lactose monohydrate;
   12 wt % to 15 wt % of talc;
   1.0 wt % to 3.0 wt % of triethyl citrate;
   0.01 wt % to 0.5 wt % of ethylcellulose; and
   0.01 wt % to 0.5 wt % of Macrogol® 6000.

Preferred are still oral formulations as described herein consisting of: a plurality of particles in form of coated granules, coated pellets, coated beads, coated minicapsules; wherein the core material is in form of a core pellet consisting of microcrystalline cellulose;
said particle consists of:
   9.80 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   39.19 wt % of microcrystalline cellulose core (Cellets 500);
   17.57 wt % of HPMCAS;
   15.58 wt % of lactose monohydrate;
   13.11 wt % of talc;
   1.67 wt % of povidone K25;
   2.76 wt % of triethyl citrate, and
   0.33 wt % of ammonium hydrogen carbonate;
or
said particle consists of:
   9.80 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   39.19 wt % of microcrystalline cellulose core (Cellets 500);
   17.57 wt % of HPMCAS;
   14.40 wt % of lactose monohydrate;
   13.11 wt % of talc;
   1.49 wt % of povidone K25;
   2.76 wt % of triethyl citrate:
   1.35 wt % of sodium dodecylsulfate; and
   0.33 wt % of ammonium hydrogen carbonate;
or
said particle consists of:
   9.68 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   38.70 wt % of microcrystalline cellulose core (Cellets 500);
   17.57 wt % of HPMCAS;
   15.38 wt % of lactose monohydrate;
   13.01 wt % of talc;
   1.64 wt % of povidone K25;
   2.76 wt % of triethyl citrate:
   0.33 wt % of ammonium hydrogen carbonate; and
   0.92 wt % of HPMC;
or
said particle consists of:
   8.89 wt % of (S,E)-methyl-7-(1-(2-(2-ethylbutylamino)-2-oxoethyl)-2-oxo-1 2-dihydro-pyridin-3-ylamino)-6-(1-methyl-1H-imidazole-5-carboxamido)-7-oxohept-2-enoate;
   34.75 wt % of microcrystalline cellulose core (Cellets 500);
   21.44 wt % of Eudragit® L100 and 3.78 wt % of Eudragit® S100;
   13.81 wt % of lactose monohydrate;
   13.55 wt % of talc; and
   2.55 wt % of triethyl citrate;
or
said particle consists of:
   7.93 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
   37.58 wt % of microcrystalline cellulose core (Cellets 500);
   24.86 wt % of Eudragit® L100;
   12.61 wt % of lactose monohydrate;
   13.01 wt % of talc;
   2.48 wt % of triethyl citrate;
   0.12 wt % of ethylcellulose; and
   0.06 wt % of Macrogol® 6000.

In some preferred embodiments, in any of the inventive oral formulations as described herein, the enteric coating polymer is dissolved at a pH-value in a range of 5.0 to 7.5, preferably 5.0 to 7.0, more preferably 5.5 to 7.0, most preferably 5.5 to 6.8.

In some preferred embodiments, in any of the inventive oral formulations as described herein, the size of each particle is in a range from 0.5 mm to 5 mm. The coated minitablets are preferably in the size of 1 mm to 4 mm.

The oral formulations in form of coated granules, coated pellets, coated beads, coated minicapsules have a size of each particle is in a range from 0.5 mm to 1.3 mm, preferably 0.6 mm-1.2 mm, more preferably 0.7 mm-1.1 mm, more preferably 0.8 mm-1.0 mm.

In some preferred embodiments, in any of the inventive oral formulations as described herein, the plurality of particles is contained in a capsule, sachet, or stick pack or is formed as a tablet. Thus, the oral formulations of the present invention is in a form of a capsule, sachet, or stick pack, or a tablet.

### Indications

Surprisingly, it was found that the inventive oral solid formulation exhibits gastro-resistance and that releases (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof in the ileum after a certain lag-time required to pass or transit the stomach and duodenum.

In order to ensure topical availability in the ileum drug release should already start in the jejunum. Gastro-resistant oral solid formulations utilizing the described release profile are also often classified as delayed release dosage forms. Delayed release dosage forms require a specific trigger for drug release such as a target pH value or enzymes. A time-dependent release is also an option to facilitate the delay. The preferred embodiment according to this invention refers to an oral solid dosage form that releases the drug in the jejunum and ileum after a certain pH value has been reached. The pH value that triggers the release should range from 6.2 to 6.8.

The oral solid formulation suitable for delayed release can comprise either single unit non-disintegrating dosage forms such as tablets or disintegrating dosage forms containing multiple units of granules/pellets/beads also known as multiple unit pellets systems or MUPS. An embodiment according to the invention is preferably a multiparticulate dosage form that provides several advantages over non-disintegrating dosage forms such as a predictable residence time in the fasted and fed stomach with a low risk of dose dumping, a reliable and robust release pattern through a homogeneous distribution of the drug over a relative high particle surface and the potential to flexibly transform the units into a variety of different dose strengths. The term granules/pellets/beads as used herein designates comparatively small spherical particles having a diameter ranging from about 0.2 to about 1.8 mm, preferably from about 0.5 to about 1.5 mm, which contain (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof and suitable excipients.

One embodiment according to the invention is related to a systemic formulation for use in the prophylaxis and/or treatment of fibrostenotic Crohn's disease containing (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof, wherein the systemic formulation is in form of an enteral formulation.

It is preferred that the enteral formulation is an oral solid formulation for tailored drug release, i.e. delivering (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof to the proximal and distal ileum. Ileal targeting refers to the property of an oral formulation to facilitate the local availability and local action of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof at a predefined segment of the small intestine avoiding premature release in the upper parts of the gastrointestinal tract (i.e. stomach and duodenum).

In some embodiments, the present invention is directed to the oral formulation of the present invention for use in the prophylaxis and/or treatment of intestinal fibrosis.

Preferably, for use in the prophylaxis and/or treatment of intestinal fibrosis, the oral formulation is adapted for starting release of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof in the jejunum and completing the release in the ileum.

Preferably, the oral formulation is useful for the prophylaxis and/or treatment of fibrostenotic Crohn's disease.

### Preparation Method

In another aspect, the present invention refers to a method for the preparation of the oral formulations as described above, comprising:
Step A) preparing a plurality of particles in form of coated granules, coated pellets, coated beads, coated minicapsules, or coated minitablets, wherein each particle contains
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I)

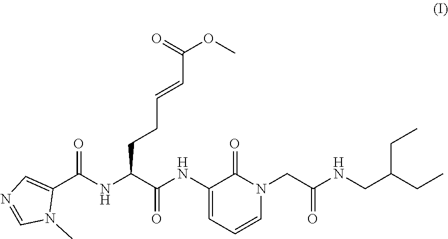 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof; and
a core material;
Step B) coating each particle with at least one coating solution comprising an enteric coating polymer.

Optionally, the preparation method further comprises the Step C):
Step C) coating each particle with a coating solution comprising sustained-release polymer.

In some embodiments, Step A)-Step B)-Step C) are performed. Alternatively, Step A)-Step C)-Step B) can also performed.

Thus, in some embodiments, the present invention refers to a method for the preparation of the oral formulations as described above, comprising:
Step A) preparing a plurality of particles in form of coated granules, coated pellets, coated beads, coated minicapsules, or coated minitablets, wherein each particle contains
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I)

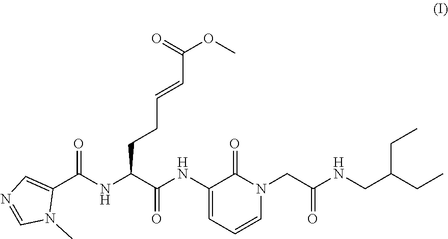 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof; and
a core material;
Step B) coating each particle with at least one coating solution comprising an enteric coating polymer; and
Step C) coating each particle with a coating solution comprising sustained-release polymer.

Alternatively, a method for the preparation of the oral formulations as described above, comprises
Step A) preparing a plurality of particles in form of coated granules, coated pellets, coated beads, coated minicapsules, or coated minitablets, wherein each particle contains
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I)

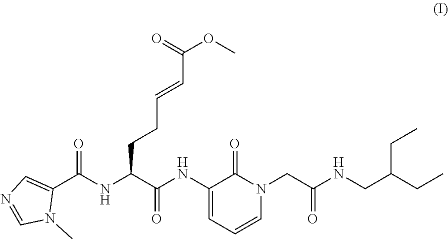 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof; and
a core material;
Step C) coating each particle with a coating solution comprising sustained-release polymer; and
Step B) coating each particle with at least one coating solution comprising an enteric coating polymer.

Preferably, in the method according to invention, wherein in case the particle is in form of a coated minitablet, the core material comprises one or more binder(s), disintegrants(s), glidant(s), pH-modifier(s), and/or filler(s). The pH-modifier may be optionally used.

More preferably, the core material comprises one or more binder(s), disintegrants(s), glidant(s), pH-modifier(s), and/or filler(s) and
the binder is low-substituted hydroxypropylcellulose, hydroxypropylcellulose, povidone K25, or a mixture thereof;
the disintegrant is croscarmellose sodium;
the glidant is talc; and/or
the filler is mannitol, lactose monohydrate, or a mixture thereof.

Still more preferably, the core material comprises one or more binder(s), disintegrants(s), glidant(s), pH-modifier(s), and/or filler(s) and
the binder is low-substituted hydroxypropylcellulose, hydroxypropylcellulose, povidone K25, or a mixture thereof:
the disintegrant is croscarmellose sodium;
the glidant is talc;
the pH-modifier is adipic acid; and/or
the filler is mannitol, lactose monohydrate, or a mixture thereof.

In another aspect, the present invention is directed to a method for preparation of an oral formulation containing a core material in form of a core pellet consisting of: tartaric acid, lactose, sugar, maize starch, starch hydrolysates, silica, or microcrystalline cellulose, preferred, microcrystalline collulose.

Hence, in some embodiments, the invention refers to a method for preparation of the oral formulation adapted for selective delivery of a drug to the small intestine of a mammal comprising or consisting of:
a plurality of particles in form of coated granules, coated pellets, coated beads, or coated minicapsules, each particle containing
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
   a core material, the core material is in form of a core pellet consisting of: tartaric acid, lactose, sugar, maize starch, starch hydroxylates, silica, or microcrystalline cellulose; and
   an enteric coating polymer,
      wherein each particle is coated with at least one coating layer containing or consisting of the enteric coating polymer.

Preferably, the core material is in form of a core pellet consisting of microcrystalline cellulose.

Thus, the invention refers to a method for preparation of said oral formulation comprising
Step A) preparing a plurality of particles in form of coated granules, coated pellets, coated beads, coated minicapsules, or coated minitablets, wherein each particle contains
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I)

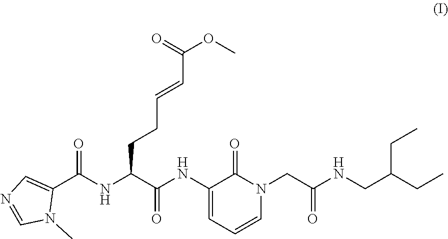 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof; and
a core material, the core material is in form of a core pellet consisting of: tartaric acid, lactose, sugar, maize starch, starch hydroxylates, silica, or microcrystalline cellulose; and
Step B) coating each particle with at least one coating solution comprising an enteric coating polymer.

Optionally, the preparation method further comprises the Step C):
Step C) coating each particle with a coating solution comprising sustained-release polymer.

In some embodiments, Step A)-Step B)-Step C) are performed. Alternatively, Step A)-Step C)-Step B) can also performed.

Thus, in some embodiments, the present invention refers to a method for the preparation of the oral formulations as described above, comprising:
Step A) preparing a plurality of particles in form of coated granules, coated pellets, coated beads, coated minicapsules, or coated minitablets, wherein each particle contains
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I)

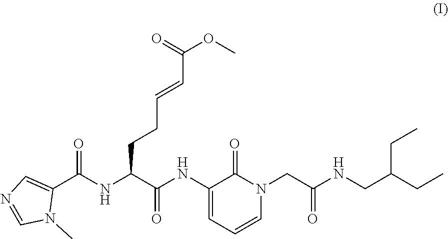 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof; and
a core material, the core material is in form of a core pellet consisting of: tartaric acid, lactose, sugar, maize starch, starch hydroxylates, silica, or microcrystalline cellulose;
Step B) coating each particle with at least one coating solution comprising an enteric coating polymer; and
Step C) coating each particle with a coating solution comprising sustained-release polymer.

Alternatively, a method for the preparation of the oral formulations as described above, comprises
Step A) preparing a plurality of particles in form of coated granules, coated pellets, coated beads, coated minicapsules, or coated minitablets, wherein each particle contains
   (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I)

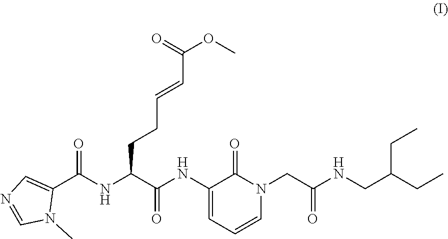 [SMILES: CCC(CC)CNC(=O)Cn1cccc(NC(=O)[C@H](CC/C=C/C(=O)OC)NC(=O)c2cncn2C)c1=O]

or an enantiomer, a solvate, a hydrate or a pharmaceutically acceptable salt thereof; and
a core material, the core material is in form of a core pellet consisting of: tartaric acid, lactose, sugar, maize starch, starch hydroxylates, silica, or microcrystalline cellulose;
Step C) coating each particle with a coating solution comprising sustained-release polymer; and
Step B) coating each particle with at least one coating solution comprising an enteric coating polymer.

Preferably, the core material is in form of a core pellet consisting of tartaric acid, lactose, sugar, maize starch, starch hydroxylates, silica, or microcrystalline cellulose which is coated with (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof, together with at least one binder(s), colorant(s), glidant(s), surfactant(s) and/or filler(s).

Hypromellose (HPMC) may firstly be coated under the coating layer containing or consisting of the enteric coating polymer. Ethylcellulose, Eudragit® RL100, Eudragit® RS100, Eudragit® RL12.5, Eudragit® RS12.5 may be subsequently coated on the coating layer containing or consisting of the enteric coating polymer.

In case the sustained-release polymer is ethylcellulose, optionally polyethylene glycol (PEG) is further comprised as plasticizer, preferably Macrogol® 6000.

Therefore, the Step A) may be replaced with the following steps A-1) and A-2) and the oral formulation of the present invention can be prepared as follows;
Step A-1) providing core material in form of a plurality of core pellets consisting of: tartaric acid, lactose, sugar, maize starch, starch hydroxylates, silica, or microcrystalline cellulose;
Step A-2) coating a solution comprising (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I), an enantiomer, a solvate, a hydrate, or a pharmaceutically acceptable salt thereof, together with at least one binder, colorant, glidant, surfactant and/or filler,
   to obtain a plurality of particles in form of coated granules, coated pellets, coated beads, coated minicapsules;
Step B) coating each particle with at least one coating solution comprising an enteric coating polymer; and optionally
Step C) coating each particle with a coating solution comprising a sustained-release polymer.

Alternatively, the oral formulation of the present invention can be prepared as follows:
Step A-1) providing core material in form of a plurality of core pellets consisting of: tartaric acid, lactose, sugar, maize starch, starch hydroxylates, silica, or microcrystalline cellulose:
Step A-2) coating a solution comprising (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I), an enantiomer, a solvate, a hydrate, or a pharmaceutically acceptable salt thereof, together with at least one binder, colorant, glidant, surfactant and/or filler,
   to obtain a plurality of particles in form of coated granules, coated pellets, coated beads, coated minicapsules;
Step C) coating each particle with a coating solution comprising a sustained-release polymer; and
Step B) coating each particle with at least one coating solution comprising an enteric coating polymer.

Preferably, in Step A-1) the core material is in form of a core pellet consisting of microcrystalline cellulose, more preferably, the particle size of the microcrystalline cellulose core pellets is range from 100 to 355 μm (Cellet 100, Cellet 200).

Therefore, the Step A) may be replaced with the following steps A-1) and A-2) and the oral formulation of the present invention can be prepared as follows:
Step A-1) providing core material in form of a plurality of core pellets consisting of microcrystalline cellulose, more preferably Cellet 100, Cellet 200, Cellet 350, or Cellet 500;
Step A-2) coating a solution comprising (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I), an enantiomer, a solvate, a hydrate, or a pharmaceutically acceptable salt thereof, together with at least one binder, colorant, glidant, surfactant and/or filler,
   to obtain a plurality of particles in form of coated granules, coated pellets, coated beads, coated minicapsules.
Step B) coating each particle with at least one coating solution comprising an enteric coating polymer; and optionally
Step C) coating each particle with a coating solution comprising a sustained-release polymer.

In some embodiments, in the method as described above,
the binder is povidone K25;
the colorant is selected from titanium dioxide, iron(III)oxide, iron(II,III) oxide, hydrated ferric oxide, lactose monohydrate, carnauba wax, and a mixture thereof;
the surfactant is sodium dodecylsulfate.
the glidant is talc; and/or
the filer is lactose monohydrate;

In some embodiments, in in Step B) of the method as described above, the one or more coating solution further comprises at least one buffering agent, plasticiser, and/or glidant.

Preferably, the buffering agent is ammonium hydrogen carbonate;
the plasticiser is triethyl citrate; and/or
the glidant is talc, silicon dioxide and a mixture thereof.

In some preferred embodiments, in Step B) of the method as described above, the enteric coating polymer is selected from the group consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

The hypromellose acetate succinate is preferably selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF; and/or the methacrylic acid-methyl methacrylate copolymer is selected from the group consisting of: Eudragit® L100, Eudragit® S100, and a mixture thereof.

In some embodiments, in in Step B) of the method as described above, water or a mixture of water and isopropyl alcohol is used for preparing the one or more coating solution(s).

In some preferred embodiments, in Step C) of the method as described above, the sustained-release polymer is selected from hypromellose (HPMC), ethylcellulose, ammonio methacrylate co-polymers such as Eudragit® RL100, Eudragit® RS100, Eudragit® RL12.5, Eudragit® RS12.5, or a mixture thereof; preferably hypromellose (HPMC).

In case the sustained-release polymer is ethylcellulose, optionally polyethylene glycol (PEG) is further comprised as plasticizer, preferably Macrogol® 6000.

In case the Step C) is performed after the Step B), the coating solution comprises the sustained-release polymer selected from ethylcellulose, ammonio methacrylate co-polymers such as Eudragit® RL100, Eudragit® RS100, Eudragit® RL12.5, Eudragit® RS12.5, or a mixture thereof; preferred, ethylcellulose, and a mixture thereof. In case the sustained-release polymer is ethylcellulose, optionally polyethylene glycol (PEG) is further comprised as plasticizer, preferably Macrogol® 6000.

In case the Step C) is performed before the Step B), the coating solution comprises hypromellose (HPMC) as the sustained-release polymer.

An embodiment according to the invention refers to delayed release granules/pellets/beads that are obtained by a continuous two-step coating process in a fluidized bed system. Inert starter granules/pellets/beads are mobilised in the fluidized bed of the coater and sprayed with the coating or feeding solutions. Inert starter granules/pellets/beads are neutral uniform spheres for coating and layering. They are available in different but reproducible sizes. Inert starter granules/pellets/beads include tartaric acid core pellets, lactose core pellets, sugar core pellets, silica core pellets and microcrystalline cellulose core pellets. Preferably, the core pellets are microcrystalline cellulose pellets. The size of the microcrystalline cellulose core pellets can range from 100 to 200 μm (Cellets 100), 200 to 355 μm (Cellets 200), 350 to 500 μm (Cellets 350), 500 to 710 μm (Cellets 500), 700 to 1000 μm (Cellets 700) and from 1000 to 1400 μm (Cellets 1000). Preferably the particle size of the microcrystalline cellulose core pellets ranges from 500 to 710 μm (Cellets 500). More preferably, the particle size of the microcrystalline cellulose core pellets ranges from 200 μm to 710 μm (Cellets 200, 350, 500), still more preferably from 350 μm to 710 μm (Cellet 350, 500), and most preferably from 500 μm to 710 μm (Cellet 500).

### DESCRIPTION OF FIGURES

FIG. 1: shows in vitro dissolution profiles of compound 1 (50 mg) from delayed release granules: Drug-layered granules with 35% HPCMAS means HPCMAS-HF and Drug-layered granules with 35% Eudragit® L/S refers to Eudragit® L100/S100).

### EXAMPLES

### Preparation Procedures

### 1. Preparation Method 1

### 1.1. Preparation of Granules/Pellets/Beads:

One or more coatings can be layered on the neutral core pellets in the fluidized bed. During this process each coating forms a thin film or layer adhering to the surface of the pellets. Each layer can provide a certain functionality to the granules/pellets/beads such as layering, sealing, protecting, release controlling, taste masking or improving ease of swallowing. In the preferred embodiment the inert core pellets are layered with (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof, a binder, a filler and a lubricant/glidant in order to obtain the drug layered pellets. The inert core pellets are sprayed with the coating or feeding solution that consists of the solute and the solvent in a preferred ratio of 25% w/w for the solute to 75% w/w for the solvent. The solute comprises a quaternary mixture of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof, povidone K25 used as a binder, lactose monohydrate used as a filler and talc used as a lubricant/glidant in a preferred ratio of about 28% for (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof, 5% for povidone K25, 45% for lactose monohydrate and 23% for talc.

The solvent of the spraying or feeding solution is a binary mixture of isopropyl alcohol and purified water in a ratio of 20% w/w (isopropyl alcohol) to 80% w/w (purified water). After the coating step the mass of the pellets has increased by approximately 90% (weight gain) and the drug (Comp. 1) concentration of the granules/pellets/beads is approximately 132 mg/g (i.e. 13.2%). A quantitative summary of the drug layered granules/pellets/beads is presented below:

| | | | |
|---|---|---|---|
| Solvent: | | Solute: | |
| Isopropyl alcohol | 20.4% | compound 1 | 28.1% |
| Purified water | 79.6% | Povidone K25 | 4.8% |
| | | Talc | 22.5% |
| | | Lactose | 44.7% |
| | | monohydrate | |
| | [symbol] | [symbol] | |

Coating solution:

| | |
|---|---|
| Compound 1 | 7.0% |
| Povidone K25 | 1.2% |
| Talc | 5.6% |
| Lactose monohydrate | 11.1% |
| Isopropyl alcohol | 58.8% |
| Purified water | 15.3% |

↓
Compound 1 granules/pellets/beads:

| | |
|---|---|
| Cellets 500 | 52.9% |
| Compound 1 | 13.2% |
| Povidone K25 | 2.2% |
| Talc | 10.6% |
| Lactose monohydrate | 21.0% |

↓

| | |
|---|---|
| Compound 1 concentration: | 13.2% or 132.2 mg/g |
| Weight gain: | 90% |

The drug layered granules/pellets/beads can be further coated with a seal layer to protect the surface of the particles against chemical or physical stress or with a layer that improves taste masking or ease of swallowing of the pellets. Usually excipients such as derivatives of cellulose, cellulose ether such as hydroxypropyl methylcellulose (HMPC), synthetic polymers, shellac, corn protein zein or other polysaccharides and amino methacrylate copolymers are used. The coating can further comprise colorants such as titanium dioxide, iron(III)oxide, iron(II,III) oxide or hydrated ferric oxide, lactose monohydrate, and or carnauba wax. Such layered granules/pellets/beads can be presented in different dose strengths by either encapsulating the required amount of pellets into a hard capsule or by filling the required amount of pellets into sachets or stick packs. These products are fast, conventional or immediate release dosage forms.

Preferably, the drug-layered granules/pellets/beads are further coated with at least one of the release-modifying polymers in order to obtain the required delayed release profile selected from the group consisting of hypromellose acetate succinate (HPMCAS), hypromellose phthalate, methacrylic acid-methyl methacrylate copolymers, methacrylic acid-ethyl acrylate copolymers, methyl acrylate-methyl methacrylate-methacrylic acid copolymers, polyvinyl acetate phthalate, cellulose acetate phthalate (CAP), shellac or mixtures thereof. The coating can further comprise buffering agents, plasticisers, glidants/lubricants and colorants such as titanium dioxide, iron(III)oxide, iron(II,III) oxide or hydrated ferric oxide, lactose monohydrate, and or carnauba wax. Especially preferred are enteric coating polymers of hypromellose acetate succinate (HPMCAS) available in different grades such as HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof, preferably HPMCAS-HF, HPMCAS-HMP, more preferably HPMCAS-HF, and methacrylic acid-methyl methacrylate copolymers either alone or in various combinations. These coatings can also further comprise buffering agents, plasticisers, glidants/lubricants and colorants such as titanium dioxide, iron(III)oxide, iron(II,III) oxide or hydrated ferric oxide, lactose monohydrate, and or carnauba wax.

### 1.2. Preparation of Enteric Polymer Coated Granules/Pellets/Beads:

### 1.2.1 Preparation of HPMCAS Coated Granules/Pellets/Beads

The drug layered pellets are coated with the coating or feeding solution that consists of the solute and the solvent in a preferred ratio of 10% w/w for the solute to 90% w/w for the solvent. The solute comprises a quaternary mixture of HPMCAS used as enteric polymer, ammonium hydrogen carbonate (NH₄HCO₃) used as buffering agent, triethyl citrate used as plasticiser and talc used as glidant/lubricant in a in a preferred ratio of about 68% for HPMCAS, 2% for ammonium hydrogen carbonate, 11% triethyl citrate and 20% for talc. The solvent of the spraying or feeding solution is purified water. After the coating step the mass of the pellets has increased by 25% to 40% (preferred 35%) and the drug (Comp. 1) concentration of the granules/pellets/beads is approximately 98 mg/g (i.e. 9.8%). The obtained particles of the embodiment have a mean diameter of about 0.9 mm. A quantitative summary of the enteric layered drug granules/pellets/beads is presented below:

| | | | |
|---|---|---|---|
| Solvent: | | Solute: | |
| Purified water | 100.0% | HPMCAS-HF | 67.8% |
| | | NH₄HCO₃ | 1.3% |
| | | Triethyl citrate | 10.6% |
| | | Talc | 20.3% |
| | [symbol] | [symbol] | |

Coating solution:

| | |
|---|---|
| HPMCAS-HF | 6.9% |
| Ammonium hydrogen | 0.1% |
| carbonate | |
| Triethyl citrate | 1.1% |
| Talc | 2.1% |
| Water | 89.9% |

↓
Enteric or delayed-release coated compound 1
granules/pellets/beads:

| | |
|---|---|
| Compound 1 | 74.1% |
| granules/pellets/beads | |
| HPMCAS-HF | 17.6% |
| NH₄HCO₃ | 0.3% |
| Triethyl citrate | 2.8% |
| Talc | 5.3% |

↓

| | |
|---|---|
| Compound 1 concentration: | 9.8% or 97.9 mg/g |
| Weight gain: | 35% |

Surprisingly, the release profile of Comp. 1 layered pellets which are coated with HPMCAS has shown to be extremely robust against potential weight gain variations of the coating but also against potential pH variations that might exist in the intestine. Within a pH window of 6.0 to 6.8 the embodiment releases the drug consistently with a very reproducible kinetic. After a lag time or delay of 5 to 10 minutes the drug release from the granules/pellets/beads is fast and reaches the plateau after another 10 minutes. Therefore, when entering the ileum 100% of compound 1 should be available for topical action.

### 1.2.2 Preparation of Eudragit® L100/S100 Coated Granules/Pellets/Beads

The drug layered pellets are coated with the coating or feeding solution that consists of the solute and the solvent in a preferred ratio of 15% w/w for the solute to 85% w/w for the solvent. The solute comprises a quaternary mixture of Eudragit® L100 and Eudragit® S100 used as enteric polymers, triethyl citrate used as plasticiser and talc used as glidant/lubricant in a in a preferred ratio of about 63% for Eudragit® L100, 10% for Eudragit® S100, 7% for triethyl citrate and 19% for talc. The solvent of the spraying or feeding solution is a binary mixture of isopropyl alcohol and purified water in a ratio of 92% w/w (isopropyl alcohol) to 8% w/w (purified water). After the coating step the mass of the pellets has increased by 45% (weight gain) and the drug concentration of the granules/pellets/beads is approximately 91 mg/g (i.e. 9.1%). The obtained particles of the embodiment have a mean diameter ranging of about 0.9. A quantitative summary of the enteric layered drug granules/pellets/beads is presented below:

| | | | |
|---|---|---|---|
| Solvent: | | Solute: | |
| Isopropyl alcohol | 92.2% | Eudragit ® L100 | 62.5% |
| Purified water | 7.8% | Eudragit ® S100 | 11.0% |
| | | Triethyl citrate | 7.3% |
| | | Talc | 19.2% |
| | [symbol] | [symbol] | |

Coating solution:

| | |
|---|---|
| Eudragit ® L100 | 9.5% |
| Eudragit ® S100 | 1.7% |
| Triethyl citrate | 1.1% |
| Talc | 2.9% |
| Isopropyl alcohol | 78.2% |
| Water | 6.6% |

↓
Enteric or delayed-release coated compound 1
granules/pellets/beads:

| | |
|---|---|
| Compound 1 | 69.0% |
| granules/pellets/beads | |
| Eudragit ® L100 | 19.4% |
| Eudragit ® S100 | 3.4% |
| Triethyl citrate | 2.3% |
| Talc | 6.0% |

↓

| | |
|---|---|
| Compound 1 concentration: | 9.1% or 91.2 mg/g |
| Weight gain: | 45% |

The use of Eudragit® L100 and Eudragit® S100 enables the particles to deliver Compound 1 to lower parts of the gastrointestinal tract. Depending on the weight gain the lag time or delay can be increased to 15 to 30 minutes. This allows for granules/pellets/beads starting the drug release in the jejunum and completing the release in the ileum. The preferred weight gain in order to adjust the required lag time ranges from 35% to 55%.

Such enteric layered drug granules/pellets/beads can be presented in different dose strengths by either encapsulating the required amount of pellets into a hard capsule or by filling the required amount of pellets into sachets or stick packs. These products are delayed release dosage forms.

### 2. Preparation Method 2

### 2.1 Preparation of Minitablets

Compound 1 is formulated into coated minitablets. While the coating options are the same as described for the spherical multiparticulates the manufacturing of the minitablets applies different operations such as blending, granulation and compression. In the preferred embodiment (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof, hydroxypropylcellulose, low-substituted, used as a binder, croscarmellose sodium used as a disintegrant and mannitol used as a filler are sieved and pre-mixed in a suitable granulator. The powder blend is wet granulated by slowly adding a solution of hydroxypropylcellulose in ethanol 96%. Then, the moistened granules are mixed until the desired granulate structure has been reached. Afterwards, the wet granules are wet-sieved and subsequently dried at 70° C.±5° C. until the required loss on drying has been reached. The dried granules are sieved and mixed with sieved adipic acid and silicon dioxide. After addition of talc the final blend is compressed into minitablets using multi-tip minitablet punches. The weight of the resulting tablets is approximately 13 mg. The qualitative and quantitative composition of the embodiment is presented below:

| | | | |
|---|---|---|---|
| Powder blend: | | Granulation liquid: | |
| Compound 1 | 11.2% | Hydroxypropylcellulose | 14.6% |
| Hydroxypropylcellulose, | 28.6% | Ethanol 96% | 85.4% |
| low-substituted | | | |
| Croscarmellose sodium | 29.8% | | |
| Mannitol | 29.8% | | |
| | [symbol] | [symbol] | |

Granulate:

| | |
|---|---|
| Compound 1 | 11.2% |
| Hydroxypropylcellulose, low- | 27.0% |
| substituted | |
| Croscarmellose sodium | 28.1% |
| Mannitol | 28.1% |
| Hydroxypropylcellulose | 5.6% |

↓
Final blend

| | |
|---|---|
| Granulate | 85.6% |
| Adipic acid | 8.7% |
| Talc | 4.8% |
| Silicon dioxide | 1.0% |

↓
Minitablets

| | |
|---|---|
| Minitablet mass: | 12.9 mg |

1 minitablet with a mass of 12.87 mg contains 1.25 mg compound 1 or a dose of 10 mg compound 1 requires 8 minitablets

### 2.2 Preparation of Enteric Polymer Coated Minitablets

As described for the granules/pellets/beads the minitablets can also be coated with HPMCAS-HF or a mixture of Eudragit® L100 and Eudragit® S100 in order to achieve the desired delayed release properties. The qualitative and quantitative composition of the film-coated minitablets is presented below.

### a) HPMCAS

| | | | |
|---|---|---|---|
| Solvent: | | Solute: | |
| Purified water | 100.0% | HPMCAS-HF | 67.6% |
| | | Ammonium | 1.5% |
| | | hydrogen | |
| | | carbonate | |
| | | Triethyl citrate | 10.6% |
| | | Talc | 20.3% |
| | [symbol] | [symbol] | |

Coating solution:

| | |
|---|---|
| HPMCAS-HF | 6.9% |
| Ammonium hydrogen carbonate | 0.2% |
| Triethyl citrate | 1.1% |
| Talc | 2.1% |
| Water | 89.8% |

↓
Enteric or delayed-release coated compound 1 mini tablets:

| | |
|---|---|
| Compound 1 mini tablets | 66.4% |
| HPMCAS-HF | 22.7% |
| Ammonium hydrogen carbonate | 0.5% |
| Triethyl citrate | 3.6% |
| Talc | 6.8% |

↓

| | |
|---|---|
| Compound 1 concentration: | 6.4% or 63.8 mg/g |
| Weight gain: | 50% |

### b) Eudragit® L100 and Eudragit® S100

| | | | |
|---|---|---|---|
| Solvent: | | Solute: | |
| Isopropyl alcohol | 92.2% | Eudragit ® L100 | 62.5% |
| Purified water | 7.8% | Eudragit ® S100 | 11.1% |
| | | Triethyl citrate | 7.2% |
| | | Talc | 19.2% |
| | [symbol] | [symbol] | |

Coating solution:

| | |
|---|---|
| Eudragit ® L100 | 9.5% |
| Eudragit ® S100 | 1.7% |
| Triethyl citrate | 1.1% |
| Talc | 2.9% |
| Isopropyl alcohol | 78.2% |
| Water | 6.6% |

↓
Enteric or delayed-release coated compound 1 mini tablets:

| | |
|---|---|
| Compound 1 mini tablets | 66.4% |
| Eudragit ® L100 | 21.0% |
| Eudragit ® S100 | 3.7% |
| Triethyl citrate | 2.4% |
| Talc | 6.5% |

↓

| | |
|---|---|
| Compound 1 concentration: | 6.4% or 63.8 mg/g |
| Weight gain: | 50% |

The minitablets can also be obtained by hot melt extrusion. In the preferred embodiment (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate or an enantiomer, a solvate, a hydrate or a pharmaceutically acceptable salt thereof, is mixed with copovidone (copolymer of 1-vinyl-2-pyrrolidone and vinyl acetate in a ratio of 6:4 by mass) and molten in a hot-melt extruder. The melt is cooled to room temperature and milled with a hammer mill. The milled extrudate is blended with croscarmellose sodium, silicon dioxide and finally magnesium stearate. The final blend is compressed into minitablets using multi-tip tablet punches.

The following examples are prepared by using any of the methods as described above.

### Example 1—Qualitative and Quantitative Composition of Compound 1 (50 mg) Delayed Release Granules Presented in Stick Packs Based on Pellets as Core Material and HPMCAS as Enteric Polymer

One stick pack with 510.28 mg delayed release granules contains:

| | | | | |
|---|---|---|---|---|
| Cellets 500 | 200.00 | mg | 39.19% | |
| Compound 1 | 50.00 | mg | 9.80% | |
| Povidone K25 | 8.50 | mg | 1.67% | |
| Lactose monohydrate | 79.50 | mg | 15.58% | |
| HPMCAS-HF | 89.64 | mg | 17.57% | Enteric |
| Ammonium hydrogen | 1.67 | mg | 0.33% | coating |
| carbonate | | | | |
| Triethyl citrate | 14.08 | mg | 2.76% | |
| Talc | 66.89 | mg | 13.11% | |
| | 510.28 | mg | 100.00% | |

### Example 2—Qualitative and Quantitative Composition of Compound 1 (50 mg) Delayed Release Granules Presented in Stick Packs Based on Pellets as Core Material and HPMCAS as Enteric Polymer Plus Sodium Docecylsulfate as Surfactant

One stick pack with 510.28 mg delayed release granules contains:

| | | | | |
|---|---|---|---|---|
| Cellets 500 | 200.00 | mg | 39.19% | |
| Compound 1 | 50.00 | mg | 9.80% | |
| Povidone K25 | 7.60 | mg | 1.49% | |
| Lactose monohydrate | 73.50 | mg | 14.40% | |
| Sodium dodecylsulfate | 6.90 | mg | 1.35% | |
| HPMCAS-HF | 89.64 | mg | 17.57% | Enteric |
| Ammonium hydrogen | 1.67 | mg | 0.33% | coating |
| carbonate | | | | |
| Triethyl citrate | 14.08 | mg | 2.76% | |
| Talc | 66.89 | mg | 13.11% | |
| | 510.28 | mg | 100.00% | |

### Example 3—Qualitative and Quantitative Composition of Compound 1 (50 mg) Delayed Release Granules Presented in Stick Packs Based on Pellets as Core Material and Hypromellose (HPMC) as Sustained-Release Polymer Plus HPMCAS as Enteric Polymer

One stick pack with 516.74 mg delayed release granules contains:

| | | | | |
|---|---|---|---|---|
| Cellets 500 | 200.00 | mg | 38.70% | |
| Compound 1 | 50.00 | mg | 9.68% | |
| Povidone K25 | 8.50 | mg | 1.64% | |
| Lactose monohydrate | 79.50 | mg | 15.38% | |
| HPMC | 4.76 | mg | 0.92% | Sustained- |
| | | | | release coating |
| HPMCAS-HF | 90.78 | mg | 17.57% | Enteric coating |
| Ammonium hydrogen | 1.69 | mg | 0.33% | |
| carbonate | | | | |
| Triethyl citrate | 14.27 | mg | 2.76% | |
| Talc | 67.24 | mg | 13.01% | |
| | 516.74 | mg | 100.00% | |

### Example 4—Qualitative and Quantitative Composition of Compound 1 (50 mg) Delayed Release Granules Presented in Stick Packs Based on Pellets as Core Material and Methacrylic Acid-Methyl Methacrylate Copolymers as Enteric Polymers

One stick pack with 575.53 mg delayed release granules contains:

| | | | | |
|---|---|---|---|---|
| Cellets 500 | 200.00 | mg | 34.75% | |
| Compound 1 | 50.00 | mg | 8.89% | |
| Povidone K25 | 8.50 | mg | 1.48% | |
| Lactose monohydrate | 79.50 | mg | 13.81% | |
| Eudragit ® L100 | 123.37 | mg | 21.44% | Enteric |
| Eudragit ® S100 | 21.77 | mg | 3.78% | coating |
| Triethyl citrate | 14.42 | mg | 2.51% | |
| Talc | 77.97 | mg | 13.55% | |
| | 575.53 | mg | 100.00% | |

### Example 5—Qualitative and Quantitative Composition of Compound 1 (50 mg) Delayed Release Granules Presented in Stick Packs Based on Pellets as Core Material and Methacrylic Acid-Methyl Methacrylate Copolymer as Enteric Polymer and Ethylcellulose as Sustained-Release Polymer

One stick pack with 575.53 mg delayed release granules contains:

| | | | | |
|---|---|---|---|---|
| Cellets 500 | 237.00 | mg | 37.58% | |
| Compound 1 | 50.00 | mg | 7.93% | |
| Povidone K25 | 8.50 | mg | 1.35% | |
| Lactose monohydrate | 79.50 | mg | 12.61% | |
| Eudragit ® L100 | 156.75 | mg | 24.86% | Enteric |
| Triethyl citrate | 15.67 | mg | 2.48% | coating |
| Talc | 82.06 | mg | 13.01% | |
| Ethylcellulose | 0.74 | mg | 0.12% | Sustained- |
| Macrogol 6000 | 0.40 | mg | 0.06% | release coating |
| | 630.62 | mg | 100.00% | |

### Example 6—Qualitative and Quantitative Composition of Compound 1 (50 mg) Delayed Release Minitablets Presented in Stick Packs Based on Minitablets as Core Material and HPMCAS as Enteric Polymer

One stick pack with 542.45 mg delayed
release minitablets contains:

| | | | | |
|---|---|---|---|---|
| Compound 1 | 50.00 | mg | 9.22% | |
| Hydroxypropylcellulose, low- | 120.00 | mg | 22.12% | |
| substituted | | | | |
| Croscarmellose sodium | 125.00 | mg | 23.04% | |
| Mannitol | 125.00 | mg | 23.04% | |
| Hydroxypropylcellulose | 15.00 | mg | 2.77% | |
| Adipic acid | 45.00 | mg | 8.30% | |
| Talc | 31.60 | mg | 5.83% | |
| Silicon dioxide | 5.00 | mg | 0.92% | |
| HPMCAS-HF | 22.00 | mg | 4.06% | Enteric |
| Ammonium hydrogen | 0.40 | mg | 0.07% | coating |
| carbonate | | | | |
| Triethyl citrate | 3.45 | mg | 0.64% | |
| | 542.45 | mg | 100.00% | |

### Example 7—Qualitative and Quantitative Composition of Compound 1 (50 mg) Delayed Release Minitablets Presented in Stick Packs Based on Minitablets as Core Material and HPMCAS as Enteric Polymer

One stick pack with 542.45 mg delayed
release minitablets contains:

| | | | | |
|---|---|---|---|---|
| Compound 1 | 50.00 | mg | 10.05% | |
| Hydroxypropylcellulose, low- | 120.00 | mg | 24.12% | |
| substituted | | | | |
| Croscarmellose sodium | 125.00 | mg | 25.13% | |
| Mannitol | 125.00 | mg | 25.13% | |
| Hydroxypropylcellulose | 15.00 | mg | 3.02% | |
| Talc | 31.60 | mg | 6.35% | |
| Silicon dioxide | 5.00 | mg | 1.01% | |
| HPMCAS-HF | 22.00 | mg | 4.42% | Enteric |
| Ammonium hydrogen | 0.40 | mg | 0.08% | coating |
| carbonate | | | | |
| Triethyl citrate | 3.45 | mg | 0.69% | |
| | 497.45 | mg | 100.00% | |

### Example 8—Qualitative and Quantitative Composition of Compound 1 (50 mg) Delayed Release Minitablets Presented in Stick Packs Based on Minitablets as Core Material and Methacrylic Acid-Methyl Methacrylate Copolymers as Enteric Polymers

One stick pack with 542.45 mg delayed
release minitablets contains:

| | | | | |
|---|---|---|---|---|
| Compound 1 | 50.00 | mg | 9.22% | |
| Hydroxypropylcellulose, low- | 120.00 | mg | 22.12% | |
| substituted | | | | |
| Croscarmellose sodium | 125.00 | mg | 23.04% | |
| Mannitol | 125.00 | mg | 23.04% | |
| Hydroxypropylcellulose | 15.00 | mg | 2.76% | |
| Adipic acid | 45.00 | mg | 8.29% | |
| Talc | 31.25 | mg | 5.76% | |
| Silicon dioxide | 5.00 | mg | 0.92% | |
| Eudragit ® L100 | 20.30 | mg | 3.74% | Enteric |
| Eudragit ® S100 | 3.60 | mg | 0.66% | coating |
| Triethyl citrate | 2.35 | mg | 0.43% | |
| | 542.-50 | mg | 100.00% | |

### Example 9—Qualitative and Quantitative Composition of Compound 1 (50 mg) Delayed Release Miniutablets Presented in Stick Packs Based on Minitablets as Core Material and Methacrylic Acid-Methyl Methacrylate Copolymers as Enteric Polymers

One stick pack with 542.45 mg delayed
release minitablets contains:

| | | | | |
|---|---|---|---|---|
| Compound 1 | 50.00 | mg | 10.05% | |
| Hydroxypropylcellulose, low- | 120.00 | mg | 24.12% | |
| substituted | | | | |
| Croscarmellose sodium | 125.00 | mg | 25.13% | |
| Mannitol | 125.00 | mg | 25.13% | |
| Hydroxypropylcellulose | 15.00 | mg | 3.02% | |
| Talc | 31.25 | mg | 6.28% | |
| Silicon dioxide | 5.00 | mg | 1.01% | |
| Eudragit ® L100 | 20.30 | mg | 4.08% | Enteric |
| Eudragit ® S100 | 3.60 | mg | 0.72% | coating |
| Triethyl citrate | 2.35 | mg | 0.47% | |
| | 497.45 | mg | 100.00% | |

### Example 10—In Vitro Dissolution Profiles of Compound 1 (50 mg) Delayed Release Granules

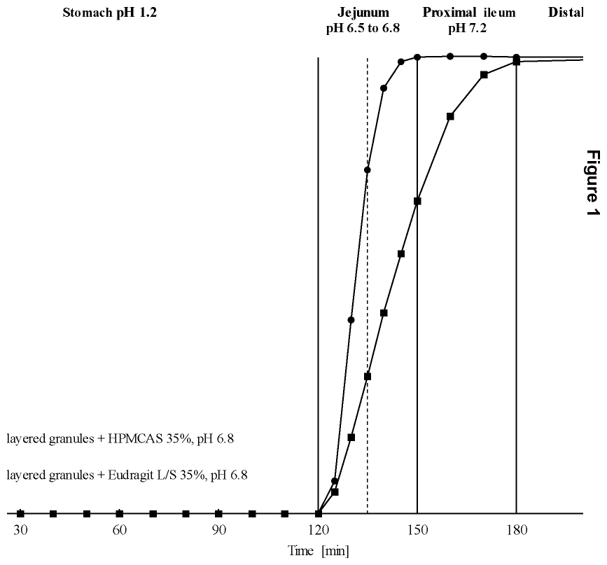

The oral formulations of the present invention can be evaluated in vitro by a dissolution test method under conditions that reflect the physicochemical characteristics of Comp. 1 and of the oral solid formulation considering the required delayed release. Based on these characteristics and considering the requirements of the European and United States Pharmacopoeia a two-stage, full change dissolution method has been developed using the paddle apparatus (i.e. apparatus II) operating with a stirring rate of 100 rpm. Gastro-resistance of the oral solid dosage form can be verified in 500 mL of simulated gastric fluid (0.1 N HCl, pH 1.2) for 2 hours. Drug release can be tested after transferring the dosage form to 900 mL of simulated intestinal fluid (50 mM phosphate buffer adjusted to a pH values of either 6.2, 6.5 or 6.8) containing 0.5% of sodium dodecylsulphate as a surfactant. The dissolution tests are conducted at 37° C. and under sink conditions to reflect in vivo conditions. An HPLC/UV method with a detection wavelength of 316 nm can be used to quantity the released amount of Comp. 1 in the dissolution media (FIG. 1.).
Apparatus: USP apparatus II
Stirring rate: 100 rpm
Dissolution media:
   Acid stage: Medium: 0.1 N HCl, pH 1.2
      Volume: 500 mL
   Buffer stage: Medium: 50 mM phosphate buffer pH 6.8 containing 0.5% sodium dodecylsulphate
      Volume: 900 mL
Sample batch number: 209702CW19 Drug-layered granules with 35% HPCMAS-HF;
   209702CW017_3 Drug-layered granules with 35% Eudragit® L100/S100
Analytical procedure: HPLC/UV
Dissolution profiles

1. An oral formulation adapted for selective delivery of a drug to the small intestine of a mammal comprising or consisting of:
a plurality of particles in form of coated granules, coated pellets, coated beads, coated minicapsules, or coated minitablets, each particle containing
(S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I): 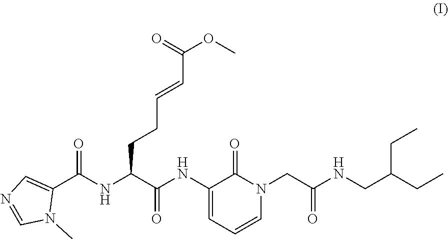 [SMILES: CCC(CC)CNC(=O)Cn1cccc(NC(=O)[C@H](CC/C=C/C(=O)OC)NC(=O)c2cncn2C)c1=O]
or an enantiomer, a solvate, a hydrate or a pharmaceutically acceptable salt thereof; a core material, and an enteric coating polymer,
wherein each particle is coated with at least one coating layer containing or consisting of the enteric coating polymer.

2. The oral formulation according to claim 1, wherein the enteric coating polymer is selected from the group comprising or consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate, hypromellose phthalate, polyvinyl acetate phthalate, cellulose acetate phthalate, shellac and mixtures of two or more of said enteric coating polymers.

3. The oral formulation according to claim 2, wherein the hypromellose acetate succinate is selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof; and/or
the methacrylic acid-methyl methacrylate copolymer is selected from the group consisting of: Eudragit® L100, Eudragit® S100, and a mixture thereof.

4. The oral formulation according to any one of the claims 1-3, wherein each particle further comprises one or more binder(s), buffering agent(s), colorant(s), plasticiser(s), glidant(s), disintegrants(s), pH-modifier(s), surfactant(s) and/or filler(s).

5. The oral formulation according to any one of the claims 1-4, wherein the enteric coating polymer is contained in or forms the outer layer of the coating enclosing the core material.

6. The oral formulation according to any one of the claims 1-5, wherein in case the particle is in form of a coated minitablet, the core material comprises one or more binder(s), disintegrants(s), glidant(s), pH-modifier(s), and/or filler(s).

7. The oral formulation according to claim 6, wherein the binder is selected from the group consisting of: sugar, sucrose, polysaccharides, xanthan gum, guar gum, carrageenan, starches derived from wheat, corn, rice and potatoes, preagglutinated starch derived from wheat, corn, rice and potatoes, sodium starch glycolate, natural gums, acacia gum, gelatin, tragacanth, derivatives of sea weed, alginic acid, sodium alginate, ammonium calcium alginate, cellulose, cellulose derivatives, hydroxypropyl cellulose, L-hydroxypropyl cellulose, low-substituted hydroxypropyl cellulose, methyl cellulose, sodium carboxymethylcellulose, hydroxypropyl methylcellulose, polyvinylpyrrolidone such as povidone K25, and mixtures thereof.

8. The oral formulation according to claim 6 or 7, wherein
the binder is low-substituted hydroxypropylcellulose, hydroxypropylcellulose, or a mixture thereof;
the disintegrant is croscarmellose sodium;
the glidant is talc; silicon dioxide, or a mixture thereof;
the pH-modifier is adipic acid; and/or
the filler is mannitol.

9. The oral formulation according to any one of the claims 1-8, wherein the at least one coating layer further comprises at least one buffering agent, plasticiser, and/or glidant.

10. The oral formulation according to claim 9, wherein
the buffering agent is ammonium hydrogen carbonate;
the plasticiser is triethyl citrate; and/or
the glidant is talc, silicon dioxide, or a mixture thereof.

11. The oral formulation according to any one of the claims 6-10, wherein each particle in form of a coated minitablet contains
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof, and
50 wt % to 90 wt % of the core material comprising or consisting of the binder, the disintegrant, the glidant, the pH-modifier and/or the filler; and
1 wt % to 10 wt % of the enteric coating polymer.

12. The oral formulation according to any one of the claims 6-11, wherein each particle comprises or consists of:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
1 wt % to 10 wt % of HPMCAS-HF;
15 wt % to 30 wt % of low-substituted hydroxypropylcellulose;
1 wt % to 10% of hydroxypropylcellulose;
15 wt % to 30 wt % of croscarmellose sodium;
15 wt % to 30 wt % of mannitol;
0.1 wt % to 3.0 wt % of triethyl citrate;
0 wt % to 1.0 wt % of ammonium hydrogen carbonate;
3 wt % to 10 wt % of talc, silicon dioxide, or a mixture thereof; and
optionally 5.0 wt % to 10.0 wt % of adipic acid;
or
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
1 wt % to 10 wt % of Eudragit® L100/S100;
15 wt % to 30 wt % of low-substituted hydroxypropylcellulose;
1 wt % to 10% of hydroxypropylcellulose;
15 wt % to 30 wt % of croscarmellose sodium;
15 wt % to 30 wt % of mannitol;
0.1 wt % to 3.0 wt % of triethyl citrate;
3 wt % 10 wt % of talc, silicon dioxide, or a mixture thereof; and
optionally 5.0 wt % to 10.0 wt % of adipic acid.

13. The oral formulation according to any one of the claims 6-12, wherein each particle consists of:
8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
3 wt % to 6 wt % of HPMCAS-HF;
20 wt % to 26 wt % of low-substituted hydroxypropylcellulose;
2 wt % to 4 wt % of hydroxypropylcellulose;
20 wt % to 26 wt % of croscarmellose sodium
20 wt % to 26 wt % of mannitol;
0.1 wt % to 1.0 wt % of triethyl citrate;
0.01 wt % to 0.5 wt % of ammonium hydrogen carbonate;
4 wt % to 8 wt % of talc;
0.5 wt % to 1.5 wt % of silicon dioxide; and
optionally, 6 wt % to 10 wt % of adipic acid.
or
each particle consists of:
8 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
3 wt % to 6 wt % of Eudragit® L100;
0.1 wt to 1.5 wt % of Eudragit® S100;
20 wt % to 26 wt % of low-substituted hydroxypropylcellulose;
2 wt % to 4 wt % of hydroxypropylcellulose;
20 wt % to 26 wt % of croscarmellose sodium
20 wt % to 26 wt % of mannitol;
0.1 wt % to 1.0 wt % of triethyl citrate;
4 wt % to 8 wt % of talc; and
0.5 wt % to 1.5 wt % of silicon dioxide; and
optionally, 6 wt % to 10 wt % of adipic acid.

14. The oral formulation according to any one of the claims 1-5, wherein in case the particle is in form of a coated granule, a coated pellet, a coated bead, or a coated minicapsule, the core material is in form of a core pellet consisting of: tartaric acid, lactose, sugar, maize starch, starch hydroxylates, silica, or microcrystalline cellulose.

15. The oral formulation according to claim 14, wherein the core pellet is coated with (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof, together with at least one binder(s), colorant(s), glidant(s), surfactant(s) and/or filler(s).

16. The oral formulation according to claim 14 or 15, wherein the size of the core pellet is in a range from 100 μm to 14000 μm.

17. The oral formulation according to any one of the claims 14-16, wherein the core pellet consists of microcrystalline cellulose and the size of the core pellet is in a range from 100 μm to 355 μm.

18. The oral formulation according to any one of the claims 15-17, wherein
the binder is polyvinylpyrrolidone;
the surfactant is sodium dodecyl sulfate; and/or
the filler is lactose monohydrate.

19. The oral formulation according to any one of the claims 14-17, wherein the layer containing the enteric coating polymer further comprises at least one buffering agent, plasticiser, and/or glidant.

20. The oral formulation according to claim 19, wherein
the buffering agent is ammonium hydrogen carbonate;
the plasticiser is triethyl citrate; and/or
the glidant is talc.

21. The oral formulation according to any one of the claims 13-20, wherein the binder is povidone K25;
the colorant is selected from titanium dioxide, iron(III)oxide, iron(II,III) oxide, hydrated ferric oxide, lactose monohydrate, carnauba wax, and a mixture thereof;
the filler is lactose monohydrate;
the buffering agent is ammonium hydrogen carbonate;
the plasticiser is triethyl citrate; and/or
the glidant is talc.

22. The oral formulation according to any one of the claims 14-21, wherein each particle is coated further with a coating layer comprising or consisting of a sustained-release polymer.

23. The oral formulation according to claim 22 wherein the sustained-release polymer is selected from hypromellose, ethylcellulose, Eudragit® RL100, Eudragit® RS100 and a mixture thereof.

24. The oral formulation according to any one of the claim 14-23, wherein each particle comprises:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof,
30 wt % to 55 wt % of the core material; and
15 wt % to 30 wt % of the enteric coating polymer.

25. The oral formulation according to any one of the claim 14-23, wherein each particle comprises:
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of formula (I), or an enantiomer, a solvate, a hydrate or a pharmaceutically acceptable salt thereof, and
−30 wt % to 55 wt % of the core material;
15 wt % to 30 wt % of the enteric coating polymer; and
0.1 wt to 2.0 wt % of the sustained-release polymer.

26. The oral formulation according to any one of the claims 14-25, wherein each said particle comprises
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
30 wt % to 45 wt % of microcrystalline cellulose core;
15 wt % to 25 wt % of HPMCAS-HF;
10 wt % 20 wt % of lactose monohydrate;
10 wt % 15 wt % of talc;
1.0 wt % to 3.0 wt % of triethyl citrate,
0.1 wt % to 1.0 wt % of ammonium hydrogen carbonate; and
optionally, 0.1 wt % to 2.0 wt % of sodium dodecylsulfate; and/or
0.1 wt to 2.0 wt % of HPMC;
or
5 wt % to 15 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
30 wt % to 45 wt % of microcrystalline cellulose core
15 wt % to 30 wt % of Eudragit® L100 and 0 wt % to 5 wt % of Eudragit® S100;
10 wt % to 20 wt % of lactose monohydrate;
10 wt % to 15 wt % of talc;
1.0 wt % to 3.0 wt % of triethyl citrate; and/or
optionally, 0.1 wt % to 2.0 wt % of ethylcellulose and PEG.

27. The oral formulation according to any one of the claims 14-26, wherein each said particle consists of:
7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
37 wt % to 41 wt % of microcrystalline cellulose core;
15 wt % to 19 wt % of HPMCAS-HF;
14 wt % to 18 wt % of lactose monohydrate;
11 wt % to 15 wt % of talc;
0.5 wt % to 2.0 wt % of povidone K25;
1.0 wt % to 3.0 wt % of triethyl citrate, and
0.1 wt % to 1.0 wt % of ammonium hydrogen carbonate;
or
said particle consists of:
7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
37 wt % to 41 wt % of microcrystalline cellulose core;
15 wt % to 19 wt % of HPMCAS-HF;
12 wt % to 16 wt % of lactose monohydrate;
11 wt % to 15 wt % of talc;
0.5 wt % to 2.0 wt % of povidone K25;
1.0 wt % to 3.0 wt % of triethyl citrate:
0.5 wt % to 2.0 wt % of sodium dodecylsulfate; and
0.1 wt % to 1.0 wt % of ammonium hydrogen carbonate;
or
said particle consists of:
7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
37 wt % to 41 wt % of microcrystalline cellulose core;
15 wt % to 19 wt % of HPMCAS-HF;
13 wt % to 17 wt % of lactose monohydrate;
11 wt % to 15 wt % of talc;
0.5 wt % to 2.0 wt % of povidone K25;
1.0 wt % to 3.0 wt % of triethyl citrate:
0.1 wt % to 1.0 wt % of ammonium hydrogen carbonate; and
0.5 wt % to 2.0 wt % of HPMC;
or
said particle consists of:
7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
33 wt % to 37 wt % of microcrystalline cellulose core;
20 wt % to 24 wt % of Eudragit® L100 and 3 wt % to 5 wt % of Eudragit® S100;
12 wt % to 16 wt % of lactose monohydrate;
12 wt % to 15 wt % of talc; and
1.0 wt % to 3.0 wt % of triethyl citrate;
or
said particle consists of:
7 wt % to 12 wt % of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate;
35 wt % to 39 wt % of microcrystalline cellulose core;
23 wt % to 27 wt % of Eudragit® L100;
10 wt % to 14 wt % of lactose monohydrate;
12 wt % to 15 wt % of talc;
1.0 wt % to 3.0 wt % of triethyl citrate;
0.01 wt % to 0.5 wt % of ethylcellulose; and
0.01 wt % to 0.5 wt % of Macrogol® 6000.

28. The oral formulation according to any one of the claims 1-27, wherein the enteric coating polymer is dissolved at a pH-value in a range of 5.5 to 6.8.

29. The oral formulation according to any one of the claims 13-28, wherein the size of each particle is in a range from 0.5 mm to 1.3 mm, preferably 0.6 mm-1.2 mm, more preferably 0.7 mm-1.1 mm, more preferably 0.8 mm-1.0 mm.

30. The oral formulation according to any one of the claims 1-29, wherein the plurality of particles is contained in a capsule, sachet, or stick pack or is formed as a tablet.

31. A oral formulation according to any one of the claims 1-30 for use in the prophylaxis and/or treatment of intestinal fibrosis.

32. The oral formulation for use according to claim 31, wherein the oral formulation is adapted for starting release of (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof in the jejunum and completing the release in the ileum.

33. The oral formulation for use according to claim 31 or 32, wherein the intestinal fibrosis is fibrostenotic Crohn's disease.

34. A method for the preparation of the oral formulation according to any one of the claims 1-30 comprising:
Step A) preparing a plurality of particles in form of coated granules, coated pellets, coated beads, coated minicapsules, or coated minitablets, wherein each particle contains
(S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate of the formula (I) 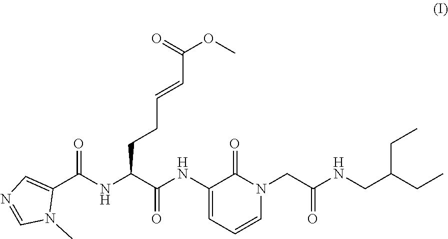 [SMILES: CCC(CC)CNC(=O)Cn1cccc(NC(=O)[C@H](CC/C=C/C(=O)OC)NC(=O)c2cncn2C)c1=O]
or an enantiomer, a solvate, a hydrate or a pharmaceutically acceptable salt thereof; and
a core material;
Step B) coating each particle with at least one coating solution comprising an enteric coating polymer.

35. The method according to claim 34, further comprising:
Step C) coating each particle with a coating solution comprising a sustained-release polymer.

36. The method according to claim 34 or 35, wherein in case the particle is in form of a coated minitablet, the core material comprises one or more binder(s), disintegrants(s), glidant(s), pH-modifier(s), and/or filler(s).

37. The method according to claim 36, wherein
the binder is low-substituted hydroxypropylcellulose, hydroxypropylcellulose, povidone K25, or a mixture thereof;
the disintegrant is croscarmellose sodium;
the glidant is talc;
the pH-modifier is adipic acid; and/or
the filer is mannitol, lactose monohydrate, or a mixture thereof.

38. The method according to claim 34 or 35, wherein the core material is in form of a core pellet consisting of tartaric acid, lactose, sugar, maize starch, starch hydroxylates, silica, or microcrystalline cellulose which is coated with (S,E)-methyl-7-(1-(2-(2-ethylbutylamino)-2-oxoethyl)-2-oxo-1,2-dihydro-pyridin-3-ylamino)-6-(1-methyl-1H-imidazole-5-carboxamido)-7-oxohept-2-enoate, an enantiomer, a solvate, a hydrate, or a pharmaceutically acceptable salt thereof, together with at least one binder(s), colorant(s), glidant(s), surfactant(s) and/or filler(s).

39. The method according to claim 38, wherein
the binder is povidone K25;
the colorant is selected from titanium dioxide, iron(III)oxide, iron(II,III) oxide, hydrated ferric oxide, lactose monohydrate, carnauba wax, and a mixture thereof;
the surfactant is sodium dodecylsulfate.
the glidant is talc; and/or
the filer is lactose monohydrate;

40. The method according to any one of the claims 34-39, wherein in Step B), the one or more coating solution further comprises at least one buffering agent, plasticiser, and/or glidant.

41. The method according to claim 40, wherein
the buffering agent is ammonium hydrogen carbonate;
the plasticiser is triethyl citrate; and/or
the glidant is talc, silicon dioxide and a mixture thereof.

42. The method according to any one of the claims 34-41, wherein in Step B) the enteric coating polymer is selected from the group consisting of: methacrylic acid-methyl methacrylate copolymers, acrylic acid-methyl methacrylate copolymers, methacrylic acid-methyl acrylate copolymers, methacrylic acid-ethyl acrylate copolymers, acrylic acid-methyl acrylate copolymers, methyl acrylate-methyl methacrylate-methacrylic acid copolymers, hypromellose acetate succinate (HPMCAS), hypromellose phthalate, polyvinyl acetate phthalate, cellulose acetate phthalate (CAP), shellac and mixtures of two or more of said enteric coating polymers.

43. The oral formulation according to claim 42, wherein the hypromellose acetate succinate is selected from the group consisting of: hypromellose acetate succinates HPMCAS-LF, HPMCAS-MF, HPMCAS-HF, HPMCAS-LMP, HPMCAS-MMP, HPMCAS-HMP, HPMCAS-LG, HPMCAS-MG, HPMCAS-HG, and a mixture thereof; and/or
the methacrylic acid-methyl methacrylate copolymer is selected from the group consisting of: Eudragit® L100, Eudragit® S100, and a mixture thereof.

44. The method according to any one of the claims 34-43, wherein in Step B), water or a mixture of water and isopropyl alcohol is used for preparing the one or more coating solution(s).
